(19) United States
(12) Patent Application Publication
(10) Pub. No.: US 2025/0032449 A1
(43) Pub. Date: Jan. 30, 2025

(54) TREATMENT OF RHEUMATOID ARTHRITIS

(71) Applicant: SYNACT PHARMA APS, Holte (DK)

(72) Inventor: Thomas Engelbrecht Nordkild JONASSEN, Holte (DK)

(21) Appl. No.: 18/713,745

(22) PCT Filed: Nov. 29, 2022
(86) PCT No.: PCT/EP2022/083624

§ 371 Date: May 28, 2024

(30) Foreign Application Priority Data
Nov. 29, 2021 (EP) 21211179.3

(51) Int. Cl. A61K 31/402; A61K 31/519; A61P 19/02

(52) U.S. Cl. CPC A61K 31/402; A61K 31/519; A61P 19/02

(57) ABSTRACT

Provided is a combination therapy comprising optimised dosages of MTX and a compound of formula (I) for the treatment of rheumatoid arthritis.

### TECHNICAL FIELD

The present invention relates to a compound of formula (I), including tautomeric and stereoisomeric forms thereof, or a pharmaceutically acceptable derivative thereof, for use in the treatment of rheumatoid arthritis, wherein said compound is to be administered with methotrexate (MTX), wherein said compound is administered at a dosage of more than 41.5 mg daily, such as at least 50 mg daily, such as at least 62 mg daily, such as at least 75 mg daily, such as once daily, and said MTX is administered at a dosage of 5 to 30 mg once weekly. This dosage regimen is observed to result in significant safety benefits qua reduced risk of adverse effects.

### BACKGROUND

An arthritic disease is a condition that implies damage or inflammation in one or more joints. The condition often presents with pain, swelling, heat, redness and limitation of movement. There are many different forms of arthritic disorders, the most common types being osteoarthritis and rheumatoid arthritis. Rheumatoid arthritis (RA) is an autoimmune disorder that primarily affects joints and between 0.5-1% of adults in the developed world are affected by RA. While the cause of rheumatoid arthritis is not clear, it is believed to involve a combination of genetic and environmental factors. The goal of current treatments is to reduce pain and inflammation to improve the quality of life of the patients suffering from the condition. Pain medications, steroids, and non-steroidal anti-inflammatory drugs (NSAIDs) are frequently used as treatment to reduce symptoms. Disease-modifying antirheumatic drugs (DMARDs), such as hydroxychloroquine and in particular methotrexate (MTX), may be employed in an attempt to slow down the progression of disease. However, MTX can be a challenge to properly dosage to avoid side effects, and not all patients respond properly to MTX (DMARD-inadequate response rheumatoid arthritis).

MTX remains the disease-modifying anti-rheumatic drug of first choice in rheumatoid arthritis (RA), but response varies. In observational studies approximately 30% of patients discontinue MTX in the medium term-around half due to inefficacy and half due to adverse events.

As an example, MTX is known to cause an increase in aminotransferases (or, transaminases). Elevated serum or plasma aminotransferases, commonly the alanine transaminase (ALT or ALAT) and aspartate transaminase (AST), may be an indicator of liver dysfunction.

Melanocortin (MC) receptors (MC1R-MC5R), a family of class A G protein-coupled receptors (GPCRs), are attractive therapeutic targets for a number of conditions due to their wide distribution and diversity of physiological processes they regulate. MC1R regulates UV light-induced skin tanning and other immune responses because of its expression on leukocytes. MC2R regulates cortisol production on the adrenal glands, whereas MC5R plays a role on exocrine glands secretions. MC3R and MC4R exert non-redundant functions on energy homeostasis in addition to specific anti-inflammatory roles; whereas MC3R activation is particularly protective for joint inflammation such as arthritis, MC4R provides neuroprotection in brain inflammation.

Peripheral MC1R and MC3R can be pharmacologically activated to induce anti-inflammation. The endogenous agonist α-melanocyte—stimulating hormone (αMSH), like other protective mediators, is released by immune cells to counterbalance proinflammatory signals, thus preventing excessive tissue damage. In line with the resolution of inflammation concept, therapeutics targeting MC1R and MC3R act by mimicking the body's own protective resources and might be characterized by a lighter burden of side effects.

Shown to be effective in rheumatic diseases since the early 1950s, the use of corticotropin or adrenocorticotropin hormone (ACTH) declined when synthetic glucocorticoids became available. However, the discovery of an alternative anti-inflammatory mechanism for ACTH involving activation of peripheral MC receptors on immune cells has revived the interest in developing novel ACTH-like molecules with no steroidogenic effects for the treatment of joint diseases such as gout or RA (rheumatoid arthritis). However, the limitation in the translational delivery of novel MC drugs besides the marketed ACTH formulations is imposed by the lack of receptor selectivity achieved so far.

Innovative approaches in G protein-coupled receptor drug discovery might help to overcome this limitation. Allosteric modulation consists in the ability of a molecule to enhance (positive modulation) or reduce (negative modulation) the effect of the endogenous ligand by binding to a distinct site of the receptor protein, termed allosteric site. A higher degree of selectivity is expected as allosteric regions are less conserved among the five MCRs, and indeed, allosteric modulators at MC4R are currently under development for the treatment of obesity.

Another emerging concept of significant therapeutic interest is the one of biased agonism. The obsolete notion that receptors could exist in two unique conformations, the active one and the inactive one, has been replaced with the conception that multiple active conformations can exist, each one creating a distinct signal yielding multiple functional outcomes. Receptor activation, rather than linear and static, is emerging as a highly dynamic and multidimensional process in which a diversity of active conformations may be induced by different molecules leading to distinct effects.

The small molecule AP1189 ((E)-N-trans-{3-[1-(2-nitrophenyl)-1H-pyrrol-2-yl]-allylidene}-aminoguanidium acetate) has been characterized as a biased agonist at receptors MC1R and MC3R, which does not induce canonical cAMP generation, but cause ERK1/2 phosphorylation, a signaling responsible for the proefferocytic effect evoked in mouse primary macrophages. AP1189 was shown to reduce cytokine release in macrophages, whereas no melanogenesis was induced by AP1189 in melanocytes. In vivo, oral AP1189 elicits anti-inflammatory actions in peritonitis and accelerated the resolution phase, and afforded significant reduction of macroscopic and histological parameters of joint disruption in experimental inflammatory arthritis. A synergistic effect of AP1189 and methotrexate (MTX) in a mouse model of arthritis has previously been demonstrated (WO 2020/229297). AP1189 is thus a biased dual agonist at MC1R and MC3R with anti-inflammatory properties together with a lack of effect on melanogenesis.

### SUMMARY

A double-blind, multi-center, two-part, randomized, placebo-controlled study of the safety, tolerability, and efficacy of treatment with AP1189 in early rheumatoid arthritis (RA) patients with active joint disease was conducted. The study population consist of newly diagnosed subjects with severe active rheumatoid arthritis (Clinical disease activity score, CDAI, >22) who are to start up-titration with methotrexate (MTX).

Patients were randomized to receive 50 mg or 100 mg AP1189 (acetate salt, corresponding to 41,5 and 83 mg AP1189 free base, respectively), or placebo.

The data presented herewith show a remarkable and dose-dependent effect of AP1189 on MTX-associated or—induced elevation of transaminases: At the 50 mg dosage AP1189 (acetate salt) did not reduce MTX-associated elevation of transaminases, while at the 100 mg dosage AP1189 was observed to completely abolish the MTX-associated elevation of transaminases, while at the same time showing efficacy in treating rheumatoid arthritis, shown as a reduction in CDAI.

It is an aspect of the present disclosure to provide a kit of parts comprising
a. more than 41.5 mg, such as at least 50 mg, such as at least 62 mg, such as at least 75 mg of a compound of formula (I):

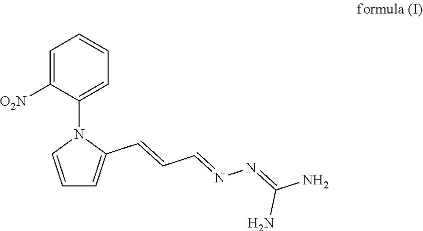 [SMILES: NC(N)=N/N=C/C=C/c1cccn1-c1ccccc1[N+](=O)[O-]]

including tautomeric and stereoisomeric forms thereof; or a pharmaceutically acceptable derivative thereof, for administration daily, such as once daily, and
b. 5 to 30 mg methotrexate (MTX), for administration once weekly, and, optionally
c. at least 5 mg folic acid, or an equivalent, per week.

It is also an aspect of the present disclosure to provide a compound of formula (I), including tautomeric and stereoisomeric forms thereof; or a pharmaceutically acceptable derivative thereof,
for use in the treatment of rheumatoid arthritis,
wherein said compound is to be administered with methotrexate (MTX),
wherein said compound is administered at a dosage of more than 41.5 mg daily, such as at least 50 mg daily, such as at least 62 mg daily, such as at least 75 mg daily, such as once daily, and said MTX is administered at a dosage of 5 to 30 mg once weekly.

It is also an aspect of the present disclosure to provide a combination comprising MTX and a compound of formula (I), including tautomeric and stereoisomeric forms thereof; or a pharmaceutically acceptable derivative thereof,
for use in the treatment of rheumatoid arthritis,
wherein said compound is administered at a dosage of more than 41.5 mg daily, such as at least 50 mg daily, such as at least 62 mg daily, such as at least 75 mg daily, such as once daily, and said MTX is administered at a dosage of 5 to 30 mg once weekly.

In some embodiments said treatment result in essentially no elevation of aminotransferases, including alanine aminotransferase (ALAT).

In some embodiments said treatment reduced or abolishes MTX-induced elevation of aminotransferases, including alanine aminotransferase (ALAT).

In some embodiments said rheumatoid arthritis is severe active rheumatoid arthritis, such as rheumatoid arthritis is rheumatoid arthritis with a CDAI>22.

In some embodiments said compound is (E)-N-trans-{3-[1-(2-nitrophenyl)-1H-pyrrol-2-yl]-allylidene}-aminoguanidine, or a pharmaceutically acceptable derivative or salt thereof.

In some embodiments said compound is (E)-N-trans-{3-[1-(2-nitrophenyl)-1H-pyrrol-2-yl]-allylidene}-aminoguanidinium acetate.

In some embodiments said compound is (E)-N-trans-{3-[1-(2-nitrophenyl)-1H-pyrrol-2-yl]-allylidene}-aminoguanidinium succinate.

### Definitions

The term “pharmaceutically acceptable derivative” in the present context includes pharmaceutically acceptable salts, which indicate a salt which is not harmful to the patient. Such salts include pharmaceutically acceptable basic or acid addition salts as well as pharmaceutically acceptable metal salts, ammonium salts and alkylated ammonium salts. A pharmaceutically acceptable derivative further includes esters and prodrugs, or other precursors of a compound which may be biologically metabolized into the active compound, or crystal forms of a compound.

The term “acid addition salt” is intended to include “pharmaceutically acceptable acid addition salt” which indicates salts which are not harmful to the patient. Acid addition salts include salts of inorganic acids as well as organic acids. Examples of pharmaceutically acceptable inorganic or organic acid addition salts include the pharmaceutically acceptable salts listed in J. Pharm. Sci. 66, 2, (1977) which is incorporated herein by reference.

Reference to AP1189 and a pharmaceutically acceptable salt of AP1189 ((E)-N-[1-(2-nitrophenyl)-1-H-pyrrole-2-yl-allylideneamino]-guanidine) including tautomeric and isomeric forms thereof, is meant to encompass amorphous forms and well as any polymorphic (crystalline) forms of AP1189 and its salts. The skilled person knows how to prepare polymorphic forms of AP1189 salts. Polymorphic forms of pharmaceutically acceptable salts of AP1189 are disclosed in PCT/EP2022/066884.

The term “therapeutically effective amount” of a compound as used herein refers to an amount sufficient to cure, alleviate, prevent, reduce the risk of, or partially arrest the clinical manifestations of a given disease or disorder and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary. ‘Amount’ and ‘dosage’ may be used interchangeably herein.

The terms “approximately” and “about” as referred herein are synonymous. In some embodiments, “approximately” and “about” refer to the recited amount, value, or duration±5%, ±4.5%, ±4%, ±3.5%, ±3%, ±2.5%, ±2%, ±1.75%, ±1.5%, ±1.25%, ±1%, ±0.9%, ±0.8%, ±0.7%, ±0.6%, ±0.5%±0.4%, ±0.3%, ±0.2%, ±0.1%, ±0.09%, ±0.08%, ±0.07%, ±0.06%, ±0.05%, ±0.04%, ±0.03%, ±0.02%, or ±0.01%. In some embodiments, “approximately” and “about” refer to the listed amount, value, or duration±2.5%, ±2%, ±1.75%, ±1.5%, ±1.25%, ±1%, ±0.9%, ±0.8%, ±0.7%, ±0.6%, ±0.5%. In some embodiments, “approximately” and “about” refer to the listed amount, value, or duration±1%. In some embodiments, “approximately” and “about” refer to the listed amount, value, or duration±0.5%. In some embodiments, “approximately” and “about” refer to the listed amount, value, or duration±0.1%.

The terms “treatment” and “treating” as used herein refer to the management and care of a patient for the purpose of combating a condition, disease or disorder. The term is intended to include the full spectrum of treatments for a given condition from which the patient is suffering. The patient to be treated is preferably a mammal, in particular a human being. Treatment of animals, such as mice, rats, dogs, cats, horses, cows, sheep and pigs, is, however, also within the scope of the present context. The patients to be treated can be of various ages.

The term “remission” as used herein refers to reduction or disappearance of the signs and symptoms of the arthritic disease. The remission can be temporary or permanent. Partial remission is a reduction in the signs and symptoms of the arthritic disease, while complete remission is understood herein as a disappearance of the signs and symptoms of the arthritic disease.

### DETAILED DESCRIPTION

The present inventors have observed that AP1189 and MTX administered in combination at certain dosages display improved properties in the treatment of rheumatoid arthritis. In particular, the risk of adverse effects are significantly reduced with an optimum dosage regimen.

### Kit of Parts

It is an aspect of the present disclosure to provide a kit of parts comprising
a. more than 41.5 mg, such as at least 50 mg, such as at least 62 mg of a compound of formula (I):

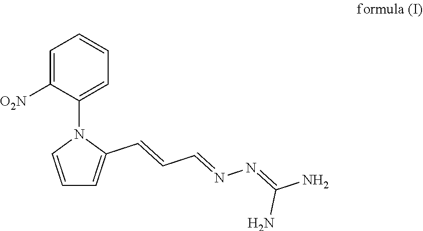 [SMILES: NC(N)=N/N=C/C=C/c1cccn1-c1ccccc1[N+](=O)[O-]]

including tautomeric and stereoisomeric forms thereof; or a pharmaceutically acceptable derivative thereof,
for administration daily, such as once daily, and
b. about 5 to about 30 mg methotrexate (MTX), for administration once weekly.

It is an aspect of the present disclosure to provide a kit of parts comprising
a. at least about 75 mg of a compound of formula (I):

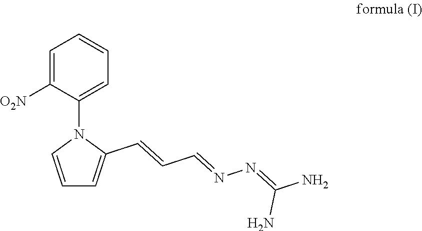 [SMILES: NC(N)=N/N=C/C=C/c1cccn1-c1ccccc1[N+](=O)[O-]]

including tautomeric and stereoisomeric forms thereof; or a pharmaceutically acceptable derivative thereof,
for administration daily, such as once daily, and
b. about 5 to about 30 mg methotrexate (MTX), for administration once weekly.

It is to be understood that the dosage of the compound of the present disclosure is calculated as the free base (not specific salt forms), unless otherwise indicated.

In some embodiments said kit of parts further comprises folic acid.

In some embodiments said kit of parts further comprises at least about 5 mg folic acid per week, such as for administration per week.

In some embodiments said kit of parts further comprises about 1 to about 10 mg folic acid per week, such as for administration per week.

In some embodiments said kit of parts further comprises about 5 mg folic acid per week, such as for administration per week.

In some embodiments said folic acid is for administration once weekly. In some embodiments said folic acid is for administration in daily dosages.

In some embodiments said kit of parts further comprises instructions for use.

In some embodiments said kit of parts is for use in the treatment of rheumatoid arthritis.

### Combination Therapy

It is an aspect of the present disclosure to provide a compound of formula (I):

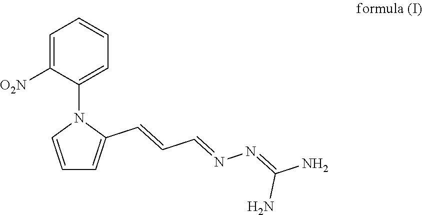 [SMILES: NC(N)=N/N=C/C=C/c1cccn1-c1ccccc1[N+](=O)[O-]]

including tautomeric and stereoisomeric forms thereof; or a pharmaceutically acceptable derivative thereof,
for use in the treatment of rheumatoid arthritis,
wherein said compound is to be administered with methotrexate (MTX),
wherein said compound is administered at a dosage of more than 41.5 mg daily, such as at least about 50 mg daily, such as at least about 62 mg daily, such as at least about 75 mg daily, such as once daily, and said MTX is administered at a dosage of about 5 to about 30 mg once weekly.

It is an aspect of the present disclosure to provide a compound of formula (I):

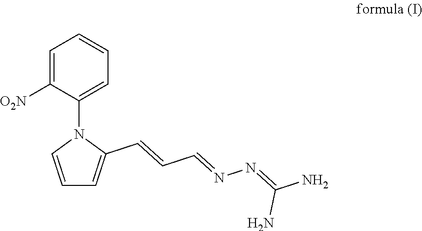 [SMILES: NC(N)=N/N=C/C=C/c1cccn1-c1ccccc1[N+](=O)[O-]]

including tautomeric and stereoisomeric forms thereof; or a pharmaceutically acceptable derivative thereof,
for use in the treatment of rheumatoid arthritis,
wherein said compound is to be administered with methotrexate (MTX),
wherein said compound is administered at a dosage of at least about 75 mg daily, such as once daily,
and said MTX is administered at a dosage of about 5 to about 30 mg once weekly.

The terms ‘is to be administered’ and ‘is administered’ may be used interchangeably herein.

It is also an aspect of the present disclosure to provide a combination of methotrexate (MTX) and a compound of formula (I):

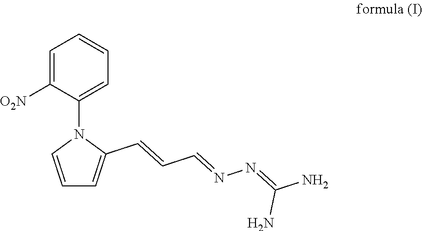 [SMILES: NC(N)=N/N=C/C=C/c1cccn1-c1ccccc1[N+](=O)[O-]]

including tautomeric and stereoisomeric forms thereof; or a pharmaceutically acceptable derivative thereof,
for use in the treatment of rheumatoid arthritis,
wherein said compound is administered at a dosage of more than 41.5 mg daily, such as at least about 50 mg daily, such as at least about 62 mg daily, such as at least about 75 mg daily, such as once daily,
and said MTX is administered at a dosage of about 5 to about 30 mg once weekly.

It is also an aspect of the present disclosure to provide a combination of methotrexate (MTX) and a compound of formula (I):

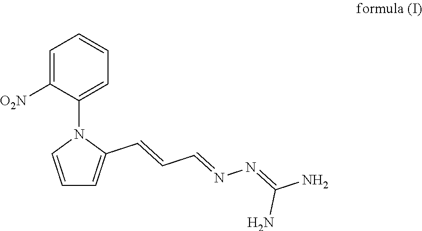 [SMILES: NC(N)=N/N=C/C=C/c1cccn1-c1ccccc1[N+](=O)[O-]]

including tautomeric and stereoisomeric forms thereof; or a pharmaceutically acceptable derivative thereof,
for use in the treatment of rheumatoid arthritis,
wherein said compound is administered at a dosage of at least about 75 mg daily, such as once daily,
and said MTX is administered at a dosage of about 5 to about 30 mg once weekly.

Also provided is a combination of methotrexate (MTX) and a compound of formula (I):

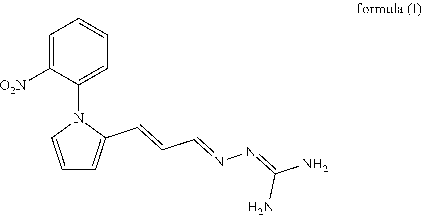 [SMILES: NC(N)=N/N=C/C=C/c1cccn1-c1ccccc1[N+](=O)[O-]]

including tautomeric and stereoisomeric forms thereof; or a pharmaceutically acceptable derivative thereof,
for the manufacture of a medicament for the treatment of rheumatoid arthritis, wherein said compound is to be administered at a dosage of more than 41.5 mg daily, such as at least about 50 mg daily, such as at least about 62 mg daily, such as at least about 75 mg daily, such as once daily,
and said MTX is to be administered at a dosage of about 5 to about 30 mg once weekly.

Also provided is a combination of methotrexate (MTX) and a compound of formula (I):

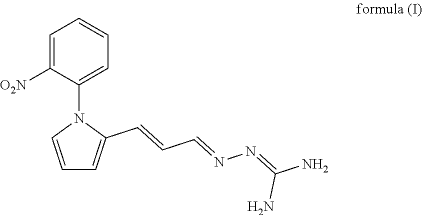 [SMILES: NC(N)=N/N=C/C=C/c1cccn1-c1ccccc1[N+](=O)[O-]]

including tautomeric and stereoisomeric forms thereof; or a pharmaceutically acceptable derivative thereof,
for the manufacture of a medicament for the treatment of rheumatoid arthritis, wherein said compound is to be administered at a dosage of at least about 75 mg daily, such as once daily,
and said MTX is to be administered at a dosage of about 5 to about 30 mg once weekly.

Also provided is a method for treating rheumatoid arthritis, said method comprising one or more steps of administering a compound of formula (I):

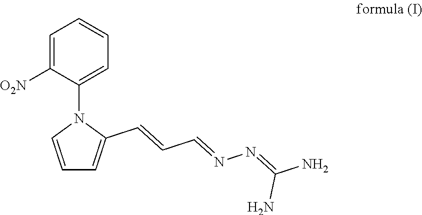 [SMILES: NC(N)=N/N=C/C=C/c1cccn1-c1ccccc1[N+](=O)[O-]]

including tautomeric and stereoisomeric forms thereof; or a pharmaceutically acceptable derivative thereof,
and further comprising one or more steps of administering methotrexate (MTX), to an individual in need thereof,
wherein said compound is administered at a dosage of more than 41.5 mg daily, such as at least about 50 mg daily, such as at least about 62 mg daily, such as at least about 75 mg daily, such as once daily,
and said MTX is administered at a dosage of about 5 to about 30 mg once weekly.

Also provided is a method for treating rheumatoid arthritis, said method comprising one or more steps of administering a compound of formula (I):

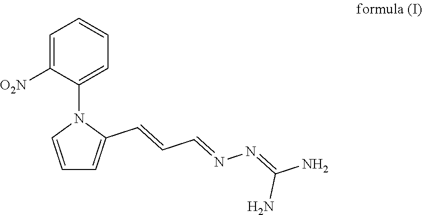 [SMILES: NC(N)=N/N=C/C=C/c1cccn1-c1ccccc1[N+](=O)[O-]]

including tautomeric and stereoisomeric forms thereof; or a pharmaceutically acceptable derivative thereof,
and further comprising one or more steps of administering methotrexate (MTX), to an individual in need thereof,
wherein said compound is administered at a dosage of at least about 75 mg daily, such as once daily,
and said MTX is administered at a dosage of about 5 to about 30 mg once weekly.

In some embodiments of the present disclosure, elevation of one or more aminotransferases is reduced or abolished.

In some embodiments of the present disclosure, MTX-induced elevation of one or more aminotransferases is reduced or abolished.

In some embodiments of the present disclosure, MTX-induced elevation of alanine aminotransferase/alanine transaminase (ALAT) is reduced or abolished.

In some embodiments of the present disclosure, elevation of alanine aminotransferase/alanine transaminase (ALAT) is reduced or abolished.

In some embodiments of the present disclosure, MTX-induced elevation of aminotransferases, such as alanine aminotransferase/alanine transaminase (ALAT), does not occur and/or is not observed.

In some embodiments of the present disclosure, no, or essentially no, elevation of aminotransferases, such as alanine aminotransferase/alanine transaminase (ALAT), occurs and/or is observed.

In some embodiments there is provided a compound of formula (I), including tautomeric and stereoisomeric forms thereof, or a pharmaceutically acceptable derivative thereof, for use in the treatment of rheumatoid arthritis, wherein said compound is to be administered with methotrexate (MTX), wherein said compound is administered at a dosage of more than 41.5 mg daily, such as at least about 50 mg daily, such as at least about 62 mg daily, such as at least about 75 mg daily, such as once daily, and said MTX is administered at a dosage of about 5 to about 30 mg once weekly, wherein elevation of one or more aminotransferases, such as ALAT, is reduced or abolished, such as wherein MTX-induced elevation of one or more aminotransferases, such as ALAT, is reduced or abolished.

In some embodiments there is provided a compound of formula (I), including tautomeric and stereoisomeric forms thereof, or a pharmaceutically acceptable derivative thereof, for use in the treatment of rheumatoid arthritis, wherein said compound is to be administered with methotrexate (MTX), wherein said compound is administered at a dosage of more than 41.5 mg daily, such as at least about 50 mg daily, such as at least about 62 mg daily, such as at least about 75 mg daily, such as once daily, and said MTX is administered at a dosage of about 5 to about 30 mg once weekly, wherein no, or essentially no, elevation of aminotransferases, such as ALAT, occurs and/or is observed; such as wherein MTX-induced elevation of aminotransferases, such as ALAT, does not occur and/or is not observed.

In some embodiments of the present disclosure, elevation of one or more aminotransferases such as MTX-induced elevation of one or more aminotransferases, such as ALAT, is less than 3%.

In some embodiments of the present disclosure, elevation of one or more aminotransferases such as MTX-induced elevation of one or more aminotransferases, such as ALAT, is less than 6%.

In some embodiments of the present disclosure, elevation of one or more aminotransferases such as MTX-induced elevation of one or more aminotransferases, such as ALAT, is about 0%, such as about 1% or less, such as about 2% or less, such as less than 3%, such as about 3% or less, such as about 4% or less, such as about 5% or less, such as less than about 6%, such as about 6% or less.

In some embodiments there is provided a compound of formula (I), including tautomeric and stereoisomeric forms thereof, or a pharmaceutically acceptable derivative thereof, for use in the treatment of rheumatoid arthritis, wherein said compound is to be administered with methotrexate (MTX),
wherein said compound is administered at a dosage of more than 41.5 mg daily, such as at least about 50 mg daily, such as at least about 62 mg daily, such as at least about 75 mg daily, such as once daily,
and said MTX is administered at a dosage of about 5 to about 30 mg once weekly, wherein elevation of one or more aminotransferases, such as ALAT; such as MTX-induced elevation of one or more aminotransferases, such as ALAT, is about 0%, such as about 1% or less, such as about 2% or less, such as less than 3%, such as about 3% or less, such as about 4% or less, such as about 5% or less, such as less than about 6%, such as about 6% or less.

Also provided is a method for reducing or abolishing MTX-induced elevation of one or more aminotransferases, such as alanine aminotransferase/alanine transaminase (ALAT), in a subject with rheumatoid arthritis, said method comprising one or more steps of administering a compound of formula (I):

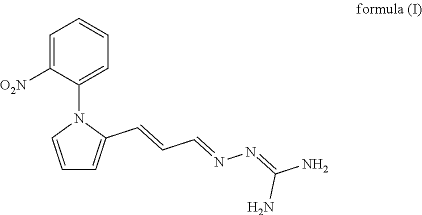 [SMILES: NC(N)=N/N=C/C=C/c1cccn1-c1ccccc1[N+](=O)[O-]]

including tautomeric and stereoisomeric forms thereof; or a pharmaceutically acceptable derivative thereof,
and further comprising one or more steps of administering methotrexate (MTX), to an individual in need thereof,
wherein said compound is administered at a dosage of more than 41.5 mg daily, such as at least about 50 mg daily, such as at least about 62 mg daily, such as at least about 75 mg daily, such as once daily,
and said MTX is to be administered at a dosage of about 5 to about 30 mg once weekly.

### Compound & MTX

It is an aspect of the present disclosure to provide a compound selected from the group consisting of {3-[1-(2-nitrophenyl)-1H-pyrrol-2-yl]-allylidene}-aminoguanidine and (E)-N-trans-{3-[1-(2-nitrophenyl)-1H-pyrrol-2-yl]-allylidene}-aminoguanidine, or a pharmaceutically acceptable salt thereof, for use in the treatment of rheumatoid arthritis,
wherein said compound is to be administered with methotrexate (MTX),
wherein said compound is administered at a dosage of more than 41.5 mg daily, such as at least about 50 mg daily, such as at least about 62 mg daily, such as at least about 75 mg daily, such as once daily,
and said MTX is administered at a dosage of 5 to 30 mg once weekly.

It is an aspect of the present disclosure to provide a compound selected from the group consisting of {3-[1-(2-nitrophenyl)-1H-pyrrol-2-yl]-allylidene}-aminoguanidine and (E)-N-trans-{3-[1-(2-nitrophenyl)-1H-pyrrol-2-yl]-allylidene}-aminoguanidine, or a pharmaceutically acceptable salt thereof, for use in the treatment of rheumatoid arthritis,
wherein said compound is to be administered with methotrexate (MTX),
wherein said compound is administered at a dosage of at least 75 mg daily, such as once daily,
and said MTX is administered at a dosage of 5 to 30 mg once weekly.

It is an aspect of the present disclosure to provide a combination of MTX and a compound selected from the group consisting of {3-[1-(2-nitrophenyl)-1H-pyrrol-2-yl]-allylidene}-aminoguanidine and (E)-N-trans-{3-[1-(2-nitrophenyl)-1H-pyrrol-2-yl]-allylidene}-aminoguanidine, or a pharmaceutically acceptable salt thereof, for use in the treatment of rheumatoid arthritis,
wherein said compound is administered at a dosage of more than 41.5 mg daily, such as at least about 50 mg daily, such as at least about 62 mg daily, such as at least about 75 mg daily, such as once daily,
and said MTX is administered at a dosage of 5 to 30 mg once weekly.

It is an aspect of the present disclosure to provide a combination of MTX and a compound selected from the group consisting of {3-[1-(2-nitrophenyl)-1H-pyrrol-2-yl]-allylidene}-aminoguanidine and (E)-N-trans-{3-[1-(2-nitrophenyl)-1H-pyrrol-2-yl]-allylidene}-aminoguanidine, or a pharmaceutically acceptable salt thereof, for use in the treatment of rheumatoid arthritis,
wherein said compound is administered at a dosage of at least 75 mg daily, such as once daily,
and said MTX is administered at a dosage of 5 to 30 mg once weekly.

In some embodiments of the present disclosure, said compound is {3-[1-(2-nitrophenyl)-1H-pyrrol-2-yl]-allylidene}-aminoguanidine, or a pharmaceutically acceptable derivative thereof.

In some embodiments of the present disclosure, said compound is (E)-N-trans-{3-[1-(2-nitrophenyl)-1H-pyrrol-2-yl]-allylidene}-aminoguanidine, or a pharmaceutically acceptable derivative thereof.

In some embodiments of the present disclosure a pharmaceutically acceptable derivative is a pharmaceutically acceptable salt.

In some embodiments of the present disclosure, said compound is (E)-N-trans-{3-[1-(2-nitrophenyl)-1H-pyrrol-2-yl]-allylidene}-aminoguanidine, or a pharmaceutically acceptable salt thereof.

In some embodiments of the present disclosure there is provided a compound (E)-N-trans-{3-[1-(2-nitrophenyl)-1H-pyrrol-2-yl]-allylidene}-aminoguanidine, or a pharmaceutically acceptable salt thereof, for use in the treatment of rheumatoid arthritis, wherein said compound is to be administered with methotrexate (MTX), wherein said compound is administered at a dosage of more than 41.5 mg daily, such as at least about 50 mg daily, such as at least about 62 mg daily, such as at least about 75 mg daily, such as once daily, and said MTX is administered at a dosage of 5 to 30 mg once weekly.

In some embodiments of the present disclosure there is provided a compound (E)-N-trans-{3-[1-(2-nitrophenyl)-1H-pyrrol-2-yl]-allylidene}-aminoguanidine, or a pharmaceutically acceptable salt thereof, for use in the treatment of rheumatoid arthritis, wherein said compound is to be administered with methotrexate (MTX), wherein said compound is administered at a dosage of at least 75 mg daily (calculated as the free base), such as once daily, and said MTX is administered at a dosage of 5 to 30 mg once weekly.

In some embodiments of the present disclosure a pharmaceutically acceptable derivative thereof is a pharmaceutically acceptable salt of an inorganic acid or an organic acid.

In some embodiments a pharmaceutically acceptable salt of an organic acid according to the present disclosure is selected from the group consisting of: formic acid, acetic acid, trichloroacetic acid, trifluoroacetic acid, propionic acid, benzoic acid, cinnamic acid, citric acid, fumaric acid, glycolic acid, lactic acid such as L-lactic acid or DL-lactic acid, maleic acid, malic acid, malonic acid, mandelic acid such as DL-mandelic acid, oxalic acid, picric acid, pyruvic acid, salicylic acid, succinic acid, methanesulfonic acid, ethanesulfonic acid, tartaric acid, ascorbic acid, pamoic acid, bismethylene salicylic acid, ethanedisulfonic acid, gluconic acid, citraconic acid, aspartic acid, stearic acid, palmitic acid, EDTA, glycolic acid, p-aminobenzoic acid, glutamic acid, benzenesulfonic acid (besylate), p-toluenesulfonic acid, hippuric acid, oxoglutaric acid such as 2-oxoglutaric acid or 3-oxoglutaric acid, glutaric acid, and adipic acid.

In a particular embodiment said organic acid is acetic acid, succinic acid, tartaric acid or propionic acid.

In a particular embodiment said organic acid is acetic acid. In a particular embodiment said organic acid is succinic acid.

In some embodiments a pharmaceutically acceptable salt of an inorganic acid according to the present disclosure is selected from the group consisting of: hydrochloric acid, hydrobromic acid, hydroiodic acid, phosphoric acid, sulphuric acid and nitric acid.

In some embodiments of the present disclosure, said compound is selected from the group consisting of:
(E)-N-[1-(2-nitrophenyl)-1-H-pyrrole-2-yl-allylideneamino]-guanidinium acetate, including tautomeric and stereoisomeric forms thereof;
(E)-N-[1-(2-nitrophenyl)-1-H-pyrrole-2-yl-allylideneamino]-guanidinium succinate including tautomeric and stereoisomeric forms thereof;
(E)-N-[1-(2-nitrophenyl)-1-H-pyrrole-2-yl-allylideneamino]-guanidine DL-mandelic acid salt including tautomeric and stereoisomeric forms thereof;
(E)-N-[1-(2-nitrophenyl)-1-H-pyrrole-2-yl-allylideneamino]-guanidine hippuric acid salt including tautomeric and stereoisomeric forms thereof;
(E)-N-[1-(2-nitrophenyl)-1-H-pyrrole-2-yl-allylideneamino]-guanidine L-lactic acid salt including tautomeric and stereoisomeric forms thereof;
(E)-N-[1-(2-nitrophenyl)-1-H-pyrrole-2-yl-allylideneamino]-guanidinium besylate including tautomeric and stereoisomeric forms thereof;
(E)-N-[1-(2-nitrophenyl)-1-H-pyrrole-2-yl-allylideneamino]-guanidinium oxoglutarate including tautomeric and stereoisomeric forms thereof;
(E)-N-[1-(2-nitrophenyl)-1-H-pyrrole-2-yl-allylideneamino]-guanidine formic acid salt including tautomeric and stereoisomeric forms thereof;
(E)-N-[1-(2-nitrophenyl)-1-H-pyrrole-2-yl-allylideneamino]-guanidine DL-lactic acid salt including tautomeric and stereoisomeric forms thereof;
(E)-N-[1-(2-nitrophenyl)-1-H-pyrrole-2-yl-allylideneamino]-guanidine glutaric acid salt including tautomeric and stereoisomeric forms thereof;
(E)-N-[1-(2-nitrophenyl)-1-H-pyrrole-2-yl-allylideneamino]-guanidine adipic acid salt including tautomeric and stereoisomeric forms thereof; and
(E)-N-[1-(2-nitrophenyl)-1-H-pyrrole-2-yl-allylideneamino]-guanidinium nitrate salt including tautomeric and stereoisomeric forms thereof.

In some embodiments of the present disclosure, said compound is selected from the group consisting of {3-[1-(2-nitrophenyl)-1H-pyrrol-2-yl]-allylidene}-aminoguanidinium acetate and (E)-N-trans-{3-[1-(2-nitrophenyl)-1H-pyrrol-2-yl]-allylidene}-aminoguanidinium acetate.

In some embodiments of the present disclosure, said compound is selected from the group consisting of {3-[1-(2-nitrophenyl)-1H-pyrrol-2-yl]-allylidene}-aminoguanidinium succinate and (E)-N-trans-{3-[1-(2-nitrophenyl)-1H-pyrrol-2-yl]-allylidene}-aminoguanidinium succinate.

In some embodiments of the present disclosure, said compound is selected from the group consisting of (E)-N-trans-{3-[1-(2-nitrophenyl)-1H-pyrrol-2-yl]-allylidene}-aminoguanidinium acetate and (E)-N-trans-{3-[1-(2-nitrophenyl)-1H-pyrrol-2-yl]-allylidene}-aminoguanidinium succinate.

In preferred embodiments of the present disclosure, said compound is selected from the group consisting of (E)-N-trans-{3-[1-(2-nitrophenyl)-1H-pyrrol-2-yl]-allylidene}-aminoguanidinium acetate and (E)-N-trans-{3-[1-(2-nitrophenyl)-1H-pyrrol-2-yl]-allylidene}-aminoguanidinium succinate.

In some embodiments of the present disclosure, said compound is (E)-N-trans-{3-[1-(2-nitrophenyl)-1H-pyrrol-2-yl]-allylidene}-aminoguanidinium acetate (‘AP1189’):

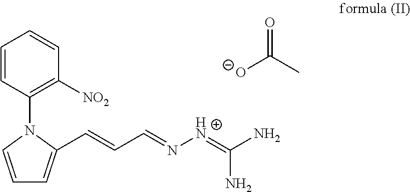

In some embodiments of the present disclosure, said compound is (E)-N-trans-{3-[1-(2-nitrophenyl)-1H-pyrrol-2-yl]-allylidene}-aminoguanidinium succinate:

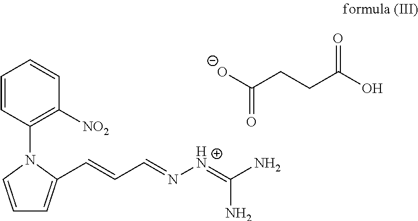

In some embodiments, the pharmaceutically acceptable salt of AP1189 according to the present disclosure is a crystalline or polymorphic form of a pharmaceutically acceptable salt of AP1189. Polymorphic forms are prepared and disclosed in PCT/EP2022/066884, the disclosure of which is incorporated by reference herewith.

In some embodiments, the pharmaceutically acceptable salt of AP1189 according to the present disclosure is a crystalline or polymorphic form of a pharmaceutically acceptable salt of AP1189 selected from the group consisting of: AP1189 acetate, AP1189 succinate, the DL-mandelic acid salt of AP1189, the hippuric acid salt of AP1189, the L-lactic acid salt of AP1189, the besylate salt of AP1189, the oxoglutarate salt of AP1189, the formic acid salt of AP1189, the DL-lactic acid salt of AP1189, the glutaric acid salt of AP1189, the adipic acid salt of AP1189 and the nitrate salt of AP1189.

In some embodiments of the present disclosure there is provided a compound selected from the group consisting of (E)-N-trans-{3-[1-(2-nitrophenyl)-1H-pyrrol-2-yl]-allylidene}-aminoguanidinium acetate (‘AP1189’) and (E)-N-trans-{3-[1-(2-nitrophenyl)-1H-pyrrol-2-yl]-allylidene}-aminoguanidinium succinate for use in the treatment of rheumatoid arthritis,
wherein said compound is to be administered with methotrexate (MTX),
wherein said compound is administered at a dosage of more than 41.5 mg daily, such as at least about 50 mg daily, such as at least about 62 mg daily, such as at least about 75 mg daily, such as once daily,
and said MTX is administered at a dosage of 5 to 30 mg once weekly.

In some embodiments of the present disclosure there is provided a compound selected from the group consisting of (E)-N-trans-{3-[1-(2-nitrophenyl)-1H-pyrrol-2-yl]-allylidene}-aminoguanidinium acetate (‘AP1189’) and (E)-N-trans-{3-[1-(2-nitrophenyl)-1H-pyrrol-2-yl]-allylidene}-aminoguanidinium succinate,
for use in the treatment of rheumatoid arthritis,
wherein said compound is to be administered with methotrexate (MTX),
wherein said compound is administered at a dosage of at least 75 mg daily (calculated as the free base), such as once daily,
and said MTX is administered at a dosage of 5 to 30 mg once weekly.

In some embodiments the present disclosure provides a compound of formula (I) as defined herein in combination with MTX for use in the treatment of rheumatoid arthritis, wherein said compound is administered at a dosage of wherein said compound is administered at a dosage of more than 41.5 mg daily, such as at least about 50 mg daily, such as at least about 62 mg daily, such as at least about 75 mg daily, such as once daily, and said MTX is administered at a dosage of 5 to 30 mg once weekly, wherein treatment with MTX and said compound is initiated essentially at the same time.

In some embodiments the present disclosure provides a compound of formula (I) as defined herein in combination with MTX for use in the treatment of rheumatoid arthritis, wherein said compound is administered at a dosage of at least 75 mg daily, such as once daily, and said MTX is administered at a dosage of 5 to 30 mg once weekly, wherein treatment with MTX and said compound is initiated essentially at the same time.

In some embodiments the subject with rheumatoid arthritis has not received MTX prior to co-treatment with the compound of the present disclosure.

In some embodiments the subject with rheumatoid arthritis is naîve to MTX treatment prior to initiating treatment with the compound of the present disclosure.

In some embodiments the subject with rheumatoid arthritis has received MTX prior to co-treatment with the compound of the present disclosure.

In some embodiments treatment with MTX is initiated prior to treatment with said compound.

In some embodiments of the present disclosure, said MTX is selected from the group consisting of methotrexate (systemic), methotrexate (oral), methotrexate tablet, methotrexate oral solution, methotrexate (injection), methotrexate sodium, Methotrexate LPF Sodium, Trexall (Xatmep), Rheumatrex, Rasuvo, Otrexup, Alltrex, Beltrax, Biotrexate, Caditrex, Carditrex, Cytotrex, Dermotrex, Folitrax, HI-Trex, Imutrex, Merex, Methocip, Methorex, Methotrexate, Metorex, Metrex, Mexate, MTX-Korea, Neotrexate, Nidtrex, Oncotrex, Onotrex, Plastomet, Remtrex, Rextop, Roxate, Tevatrex, Throtex, Trex, Thixilem, Vibzi and Zexate.

### Folic Acid

In some embodiments of the present disclosure there is provided a compound as disclosed herein, for use in the treatment of rheumatoid arthritis, wherein said compound is to be administered with methotrexate (MTX), wherein said compound is administered at a dosage of wherein said compound is administered at a dosage of more than 41.5 mg daily, such as at least about 50 mg daily, such as at least about 62 mg daily, such as at least about 75 mg daily, such as once daily, and said MTX is administered at a dosage of 5 to 30 mg once weekly, and wherein said compound is to be administered with folic acid, or an equivalent thereof.

In some embodiments of the present disclosure there is provided a compound as disclosed herein, for use in the treatment of rheumatoid arthritis, wherein said compound is to be administered with methotrexate (MTX),
wherein said compound is administered at a dosage of at least 75 mg daily, such as once daily, and said MTX is administered at a dosage of 5 to 30 mg once weekly, and wherein said compound is to be administered with folic acid, or an equivalent thereof.

In some embodiments said folic acid is administered at about 1 to 10 mg folic acid per week, such as about 5 mg folic acid per week, such as at least 5 mg folic acid per week.

In some embodiments of the present disclosure there is provided a combination comprising MTX, folic acid and a compound as disclosed herein, for use in the treatment of rheumatoid arthritis, wherein said compound is to be administered with methotrexate (MTX),
wherein said compound is administered at a dosage of more than 41.5 mg daily, such as at least about 50 mg daily, such as at least about 62 mg daily, such as at least about 75 mg daily, such as once daily,
said MTX is administered at a dosage of 5 to 30 mg once weekly,
and said folic acid is administered at a dosage of at least 5 mg folic acid per week.

In some embodiments of the present disclosure there is provided a combination comprising MTX, folic acid and a compound as disclosed herein, for use in the treatment of rheumatoid arthritis, wherein said compound is to be administered with methotrexate (MTX),
wherein said compound is administered at a dosage of at least 75 mg daily, such as once daily,
said MTX is administered at a dosage of 5 to 30 mg once weekly, and said folic acid is administered at a dosage of at least 5 mg folic acid per week.

In some embodiments said folic acid is administered once weekly.

In some embodiments said folic acid is administered in daily dosages.

### Dosages

### AP1189

In some embodiments of the present disclosure there is provided a compound as disclosed herein, for use in the treatment of rheumatoid arthritis, wherein said compound is to be administered with methotrexate (MTX), and wherein said compound is administered at a dosage of more than 41.5 mg daily, such as once daily, wherein said dosage is calculated as the free base.

A dosage of 41.5 mg free base corresponds to about 50 mg of the acetate salt of the present compound.

In some embodiments said compound is administered at a daily dosage of at least 42 mg, such as at least 43 mg, such as at least 44 mg, such as at least about 45 mg, such as at least about 46 mg, such as at least about 47 mg, such as at least about 48 mg, such as at least about 49 mg (calculated as the free base).

In some embodiments of the present disclosure there is provided a compound as disclosed herein, for use in the treatment of rheumatoid arthritis, wherein said compound is to be administered with methotrexate (MTX), and wherein said compound is administered at a dosage of at least about 50 mg daily, such as once daily, wherein said dosage is calculated as the free base.

A dosage of about 50 mg free base corresponds to about 60 mg of the acetate salt.

In some embodiments said compound is administered at a daily dosage of at least about 50 mg, such as at least about 55 mg, such as at least about 60 mg, such as at least about 62 mg, such as at least about 65 mg, such as at least about 70 mg, such as administered at a daily dosage of at least about 75 mg (calculated as the free base).

In some embodiments said compound is administered at a daily dosage of about 50 mg or more (calculated as the free base).

In some embodiments said compound is administered at a daily dosage of 42 to 45 mg, such as 45 to 50 mg, such as 50 to 55 mg, such as 55 to 60 mg, such as 60 to 62 mg, such as 62 to 65 mg, such as 65 to 70 mg, such as 70 to 75 mg (calculated as the free base).

In some embodiments of the present disclosure there is provided a compound as disclosed herein, for use in the treatment of rheumatoid arthritis, wherein said compound is to be administered with methotrexate (MTX), and wherein said compound is administered at a dosage of at least about 75 mg daily, such as once daily.

In some embodiment said compound is administered at a dosage of at least about 75 mg daily, such as once daily, wherein said dosage is calculated as the acetate salt (corresponding to about 62 mg free base).

In some embodiment said compound is administered at a dosage of at least about 75 mg daily, such as once daily, wherein said dosage is calculated as the free base (corresponding to about 90 mg of the acetate salt).

In some embodiments said compound is administered at a daily dosage of about 75 mg or more.

In some embodiments the compound is {3-[1-(2-nitrophenyl)-1H-pyrrol-2-yl]-allylidene}-aminoguanidine, for example (E)-N-trans-{3-[1-(2-nitrophenyl)-1H-pyrrol-2-yl]-allylidene}-aminoguanidine, or a pharmaceutically acceptable salt thereof, such as (E)-N-trans-{3-[1-(2-nitrophenyl)-1H-pyrrol-2-yl]-allylidene}-aminoguanidinium acetate or (E)-N-trans-{3-[1-(2-nitrophenyl)-1H-pyrrol-2-yl]-allylidene}-aminoguanidinium succinate.

The dosage used herewith is calculated as AP1189 free base, unless otherwise indicated. For example, 100 mg (E)-N-trans-{3-[1-(2-nitrophenyl)-1H-pyrrol-2-yl]-allylidene}-aminoguanidinium acetate, as is used in the Examples, corresponds to about 83 mg (E)-N-trans-{3-[1-(2-nitrophenyl)-1H-pyrrol-2-yl]-allylidene}-aminoguanidine (free base).

For the sake of clarity, a formulation comprising about 100 mg AP1189 will comprise about 120 mg AP1189 acetate.

For the sake of clarity, a formulation comprising about 100 mg AP1189 will comprise about 140 mg AP1189 succinate.

In some embodiments said compound is administered at a dosage of at least about 75 mg daily (calculated as the free base). In some embodiments said compound is administered at a daily dosage of at least about 75 mg. These terms are used interchangeably herein.

In some embodiments said compound is administered at a dosage of at least 75 mg daily, which dosage is administered in a once daily dosage.

In some embodiments said compound is administered at a dosage of more than 41.5 mg once daily, such as at least about 50 mg once daily, such as at least about 62 mg once daily, such as at least about 75 mg once daily.

In some embodiments said compound is administered at a dosage of at least 75 mg once daily.

In some embodiments said compound is administered at a dosage of more than 41.5 mg daily, such as at least about 50 mg daily, such as at least about 62 mg daily, such as at least about 75 mg daily, which dosage is divided into a twice daily or three times daily dosage.

In some embodiments said compound is administered at a dosage of at least 75 mg daily, which dosage is divided into a twice daily or three times daily dosage.

In some embodiments said compound is administered at a dosage of at least 83 mg daily (calculated as the free base), such as once daily.

In some embodiments said compound is administered at a dosage of about 83 mg daily (calculated as the free base), such as once daily.

In some embodiments said compound is (E)-N-trans-{3-[1-(2-nitrophenyl)-1H-pyrrol-2-yl]-allylidene}-aminoguanidinium acetate, and is administered at a dosage of at least 100 mg daily (calculated as the acetate salt form), such as once daily.

In some embodiments the compound of the present disclosure is administered in a dosage of more than 41.5 mg per day to about 250 mg per day (calculated as the free base), such as about 42 to about 45 mg per day, about 45 to about 50 mg per day, about 50 to about 55 mg, about 55 to about 60 mg, about 60 to about 62 mg, about 62 to about 65 mg, about 65 to about 70 mg, such as about 70 to about 75 mg per day, about 75 to about 80 mg, about 80 to about 83 mg, about 83 to about 85 mg, about 85 to about 90 mg, about 90 to about 95 mg, about 95 to about 100 mg, about 100 to about 125 mg, about 125 to about 150 mg, about 150 to about 166 mg, about 166 to about 175 mg, about 175 to about 200 mg, such as about 200 to about 250 mg per day (calculated as the free base).

In some embodiments the compound of the present disclosure is administered in an amount or dosage of about 75 mg to about 300 mg per day (calculated as the free base), such as about 75 to about 100 mg, about 100 to about 125 mg, about 125 to about 150 mg, about 150 to about 200 mg, about 200 to about 225 mg, about 225 to about 250 mg, such as about 275 to about 300 mg per day.

In some embodiments the compound of the present disclosure is administered at a dosage of about 50 mg to about 150 mg per day (calculated as the free base), such as about 50 to about 100 mg, about 100 to about 125 mg, about 125 to about 150 mg per day.

In some embodiments the compound of the present disclosure is administered in an amount or dosage of about 83 mg to about 300 mg per day (calculated as the free base), such as about 83 to about 100 mg, about 100 to about 125 mg, about 125 to about 150 mg, about 150 to about 200 mg, about 200 to about 225 mg, about 225 to about 250 mg, such as about 275 to about 300 mg per day.

In some embodiments the compound of the present disclosure is administered at a dosage of more than 41.5 mg daily to about 250 mg per day; such as at least about 50 mg daily to about 250 mg per day; such as at least about 62 mg daily to about 250 mg per day; such as at least about 75 mg daily to about 250 mg per day (calculated as the free base).

In some embodiments the compound of the present disclosure is administered at a dosage of more than 41.5 mg daily to about 166 mg per day; such as at least about 50 mg daily to about 166 mg per day; such as at least about 62 mg daily to about 166 mg per day; such as at least about 75 mg daily to about 166 mg per day (calculated as the free base).

In some embodiments the compound of the present disclosure is administered in an amount or dosage of about 75 mg once daily, such as about 83 mg once daily, such as about 100 mg once daily, such as about 150 mg once daily, such as about 166 mg once daily, such as about 200 mg once daily, such as about 249 mg once daily, such as about 250 mg once daily, such as about 300 mg once daily (calculated as the free base).

MTX

In some embodiments of the present disclosure, methotrexate (MTX) is administered in an amount of about 5 mg to about 30 mg once weekly.

In some embodiments of the present disclosure, methotrexate (MTX) is administered at a dosage of about 5 mg to about 30 mg once weekly.

In some embodiments of the present disclosure, the methotrexate (MTX) is administered at a dosage of about 5 mg once weekly, such as about 7.5 mg once weekly, such as about 10 mg once weekly, such as about 15 mg once weekly, such as about 20 mg once weekly, such as about 25 mg once weekly, such as about 30 mg once weekly.

In some embodiments of the present disclosure, the methotrexate (MTX) is administered in an amount or dosage of 5 to 5.5 mg/week, such as 5.5 to 6, such as 6 to 6.5, such as 6.5 to 7, such as 7 to 7.5, such as 7.5 to 8.5, such as 8.5 to 9, such as 9 to 9.5, such as 9.5 to 10, such as 10 to 10.5, such as 10.5 to 11, such as 11 to 11.5, such as 11.5 to 12, such as 12 to 12.5, such as 12.5 to 13, such as 13 to 13.5, such as 13.5 to 14, such as 14 to 14.5, such as 14.5 to 15, such as 15 to 15.5, such as 15.5 to 16, such as 16 to 16.5, such as 16.5 to 17, such as 17 to 17.5 such as 17.5 to 18, such as 18 to 18.5 such as 18.5 to 19, such as 19 to 19.5, such as 19.5 to 20, such as 20 to 20.5, such as 20.5 to 21, such as 21 to 21.5, such as 21.5 to 22, such as 22 to 22.5, such as 22.5 to 23, such as 23 to 23.5, such as 23.5 to 24, such as 24 to 24.5, such as 24.5 to 25, such as 25 to 25.5, such as 25.5 to 26, such as 26 to 26.5, such as 26.5 to 27, such as 27 to 27.5, such as 27.5 to 28, such as 28 to 28.5, such as 28.5 to 29, such as 29 to 29.5, such as 29.5 to 30 mg/week, preferably once weekly.

In some embodiments of the present disclosure, the methotrexate (MTX) is administered in a weekly dosage or amount of about 5 mg to about 10 mg, such as about 5 mg to about 15 mg, such as about 10 mg to about 15 mg, such as about 5 mg to about 20 mg, such as about 10 to about 20 mg, such as about 10 to about 25 mg, such as about 15 to about 20 mg.

In some embodiments of the present disclosure, the methotrexate (MTX) is administered in a weekly dosage of about 5 mg to about 10 mg, such as about 10 mg to about 15 mg, such as about 15 mg to about 20 mg, such as about 20 mg to about 25 mg, such as about 25 to about 30 mg.

In some embodiments of the present disclosure, the methotrexate (MTX) or prodrug thereof is administered subcutaneously in an amount or dosage of about 5 to about 30 mg once per week/weekly. Once per week means once every 7 days, preferably on the same week day.

In some embodiments of the present disclosure, the methotrexate (MTX) or prodrug thereof is administered in an oral dosage form in an amount or dosage of about 10 to about 25 mg once per week/weekly. Once per week means once every 7 days, preferably on the same week day.

In a preferred embodiment MTX or a prodrug thereof is administered once weekly. Commercially available dosage forms of MTX are available, for oral administration or subcutaneous administration. Both dosage forms are to be administered once a week, preferably on the same week day.

### Subject

In some embodiments the present disclosure provides a compound of formula (I), including tautomeric and stereoisomeric forms thereof, or a pharmaceutically acceptable derivative thereof, for use in the treatment of rheumatoid arthritis, wherein said compound is to be administered with methotrexate (MTX), wherein said compound is administered at a dosage of more than 41.5 mg daily, such as at least about 50 mg daily, such as at least about 62 mg daily, such as at least about 75 mg daily, such as once daily, and said MTX is administered at a dosage of 5 to 30 mg once weekly.

In some embodiments the present disclosure provides a combination of MTX and a compound of formula (I), including tautomeric and stereoisomeric forms thereof, or a pharmaceutically acceptable derivative thereof, for use in the treatment of rheumatoid arthritis, wherein said compound is administered at a dosage of more than 41.5 mg daily, such as at least about 50 mg daily, such as at least about 62 mg daily, such as at least about 75 mg daily, such as once daily, and said MTX is administered at a dosage of 5 to 30 mg once weekly.

In some embodiments the present disclosure provides a compound of formula (I) as defined herein in combination with MTX for use in the treatment of rheumatoid arthritis, wherein said compound is administered at a dosage of more than 41.5 mg daily, such as at least about 50 mg daily, such as at least about 62 mg daily, such as at least about 75 mg daily, such as once daily, and said MTX is administered at a dosage of 5 to 30 mg once weekly.

In some embodiments, the rheumatoid arthritis is severe active rheumatoid arthritis (Clinical disease activity score, CDAI>22). In some embodiments, the rheumatoid arthritis is rheumatoid arthritis with a CDAI>22.

In some embodiments the present disclosure provides a compound of formula (I) as defined herein in combination with MTX for use in the treatment of severe active rheumatoid arthritis, such as rheumatoid arthritis with a CDAI>22, wherein said compound is administered at a dosage of more than 41.5 mg daily, such as at least about 50 mg daily, such as at least about 62 mg daily, such as at least about 75 mg daily, such as once daily, and said MTX is administered at a dosage of 5 to 30 mg once weekly.

In some embodiments, the rheumatoid arthritis is early rheumatoid arthritis with active joint disease.

In some embodiments the present disclosure provides a compound of formula (I) as defined herein in combination with MTX for use in the treatment of early rheumatoid arthritis with active joint disease,
wherein said compound is administered at a dosage of more than 41.5 mg daily, such as at least about 50 mg daily, such as at least about 62 mg daily, such as at least about 75 mg daily, such as once daily,
and said MTX is administered at a dosage of 5 to 30 mg once weekly.

In some embodiments the present disclosure provides a compound of formula (I) as defined herein in combination with MTX for use in the treatment of newly diagnosed subjects with severe active rheumatoid arthritis (CDAI>22) who are to start up-titration with MTX, wherein said compound is administered at a dosage of more than 41.5 mg daily, such as at least about 50 mg daily, such as at least about 62 mg daily, such as at least about 75 mg daily, such as once daily, and said MTX is administered at a dosage of 5 to 30 mg once weekly.

In some embodiments the present disclosure provides a compound of formula (I) as defined herein in combination with MTX for use in the treatment of newly diagnosed subjects with rheumatoid arthritis (RA) with an inadequate response (IR) to MTX therapy,
wherein said compound is administered at a dosage of more than 41.5 mg daily, such as at least about 50 mg daily, such as at least about 62 mg daily, such as at least about 75 mg daily, such as once daily,
and said MTX is administered at a dosage of 5 to 30 mg once weekly.

In some embodiments the present disclosure provides a compound of formula (I) as defined herein in combination with MTX for use in the treatment of rheumatoid arthritis in a subject with an inadequate response to MTX therapy,
wherein said compound is administered at a dosage of more than 41.5 mg daily, such as at least about 50 mg daily, such as at least about 62 mg daily, such as at least about 75 mg daily, such as once daily,
and said MTX is administered at a dosage of 5 to 30 mg once weekly.

In some embodiments, the rheumatoid arthritis is rheumatoid arthritis with a DAS28 score of above 5.1.

In some embodiments the present disclosure provides a compound of formula (I) as defined herein in combination with MTX for use in the treatment of rheumatoid arthritis with a DAS28 score of above 5.1, wherein said compound is administered at a dosage of more than 41.5 mg daily, such as at least about 50 mg daily, such as at least about 62 mg daily, such as at least about 75 mg daily, such as once daily, and said MTX is administered at a dosage of 5 to 30 mg once weekly.

In some embodiments the present disclosure provides a compound of formula (I) as defined herein in combination with MTX for use in the treatment of DMARD-inadequate response rheumatoid arthritis, wherein said compound is administered at a dosage of more than 41.5 mg daily, such as at least about 50 mg daily, such as at least about 62 mg daily, such as at least about 75 mg daily, such as once daily, and said MTX is administered at a dosage of 5 to 30 mg once weekly.

In some embodiments, the subject with rheumatoid arthritis tests positive for rheumatoid factor and/or anti-cyclic citrullinated peptide (CCP) IgG antibodies prior to the treatment.

### Subjects and Assessment of Disease Severity

The subject to be treated is preferably a mammal, in particular a human being. Treatment of animals, such as mice, rats, dogs, cats, horses, cows, sheep and pigs, is, however, also within the scope of the present disclosure. The subject to be treated can be of various ages.

The severity of rheumatoid arthritis (RA), as well as the efficacy of medical treatment, of the subject can be assessed by a number of different clinical score systems, e.g. the DAS28 score, the CDAI score and the ACR-score.

For example, the change in one or more clinical scores after treatment can be evaluated by assessing the resulting chance in said clinical scores, and comparing same to baseline or placebo.

There are a wide range of measures of disease activity in RA including:
examination of the subject's joints for swelling and tenderness (Swollen Joint Count, SJC, and Tender Joint Count, TJC),
global scores of pain and overall status of the subject,
blood markers of inflammation (e.g. Erythrocyte sedimentation rate (ESR or sed rate) that indirectly measures the degree of inflammation present in the body of the subject, and the c-reactive protein test that measures the level of c-reactive protein (CRP) in the blood of the subject)
questionnaires (e.g. the Health Assessment Questionnaire Disability Index (HAQ-DI, which assesses subject function) & Functional Assessment of Chronic Illness Therapy-Fatigue (FACIT-Fatigue)),
Imaging techniques such as X-ray imaging, ultrasound imaging, and magnetic resonance imaging (MRI).

In some embodiments, the use according to the present disclosure results in improved physical function in the subject as determined by the Health Assessment Questionnaire Disability Index (HAQ-DI).

In some embodiments, the use according to the present disclosure results in improved function in the subject as determined by the Functional Assessment of Chronic Illness Therapy-Fatigue (FACIT-Fatigue).

In some embodiments, the use according to the present disclosure results in partial or complete remission of one or more arthritis symptoms.

In some embodiments, the use according to the present disclosure reduces joint inflammation.

In some embodiments, the use according to the present disclosure reduces the level of c-reactive protein (CRP) in the blood.

In some embodiments, the use according to the present disclosure reduces the number of tender joints and/or reduces the number of swollen joints.

### DAS28 Score

The DAS28 score is a measure of disease activity in rheumatoid arthritis (RA). DAS stands for ‘disease activity score’ and the number 28 refers to the 28 joints that are examined in this assessment.

The DAS28 is a composite score derived from 4 of the above measures. This ‘28’ version is a simplification of the original DAS score, which requires 44 joints to be counted. Other versions of the DAS28 allow the CRP to be used instead of the ESR, or the omission of either. The DAS28-CRP, part of the many DAS scores for RA, is very useful to make an objective, reproducible and comparable assessment of the rheumatoid arthritis activity. DAS28-CRP in particular takes into account the following items:
TJC28: The number of tender joints (0-28).
SJC28: The number of swollen joints (0-28).
CRP: The C-Reactive Protein level (in mg/l).
GH: The patient global health assessment (from 0=best to 100=worst).

The 28 tender or swollen joint scores target the same joints (shoulders, elbows, wrists, metacarpophalangeal joints, proximal interphalangeal joints and the knees). The computation of the score can be done through the following equation:

DAS28_{CRP}=0.56*\sqrt{TJC28}+0.28*\sqrt{SJC28}+0.36*ln(CRP+1)+0.014*GH+0.96

Generally, remission is considered achieved if the score is between 0 and <2.6. Low disease activity corresponds to 2.6 to <3.2. Moderate disease activity is between 3.2 and ≤5.1, while high disease activity is strictly above 5.1.

In some embodiments the present disclosure provides a compound of formula (I) as defined herein in combination with MTX for use in the treatment of rheumatoid arthritis, wherein said compound is administered at a dosage of at least 75 mg daily, such as once daily, and said MTX is administered at a dosage of 5 to 30 mg once weekly, wherein the Disease Activity Score 28 (DAS28) is determined for the subject prior to and after treatment, wherein the treatment results in a reduced DAS28 score, such as wherein the treatment results in a DAS28 score below 5.1, such as 5.0 or less, such as 4.8 or less, such as 4.6 or less, such as 4.4 or less, such as 4.2 or less, such as 4.0 or less, such as 3.8 or less, such as 3.6 or less, such as 3.4 or less, such as 3.2 or less, such as 3.0 or less, such as 2.8 or less, such as 2.6 or less, such as 2.4 or less, such as 2.2 or less, such as 2.0 or less, such as 1.8 or less, such as 1.6 or less, such as wherein the treatment results in a DAS28 score below 5.1, preferably 3.2 or less, more preferably 2.6 or less.

In some embodiments said subject's DAS28 score during and/or after treatment is reduced to between 3.2 and ≤5.1 (moderate activity), such as reduced to between 2.6 to <3.2 (low activity), such as reduced to between 0 and <2.6 (remission).

In some embodiments the present disclosure provides a compound of formula (I) as defined herein in combination with MTX for use in the treatment of rheumatoid arthritis in a subject with a DAS28 score of above 5.1, wherein said compound is administered at a dosage of more than 41.5 mg daily, such as at least about 50 mg daily, such as at least about 62 mg daily, such as at least about 75 mg daily, such as once daily, and said MTX is administered at a dosage of 5 to 30 mg once weekly.

In some embodiments the present disclosure provides a compound of formula (I) as defined herein in combination with MTX for use in the treatment of rheumatoid arthritis in a subject with a DAS28 score of between 3.2 and ≤5, wherein said compound is administered at a dosage of more than 41.5 mg daily, such as at least about 50 mg daily, such as at least about 62 mg daily, such as at least about 75 mg daily, such as once daily, and said MTX is administered at a dosage of 5 to 30 mg once weekly.

In some embodiments the present disclosure provides a compound of formula (I) as defined herein in combination with MTX for use in the treatment of rheumatoid arthritis in a subject with a DAS28 score of between 2.6 to <3.2, wherein said compound is administered at a dosage of more than 41.5 mg daily, such as at least about 50 mg daily, such as at least about 62 mg daily, such as at least about 75 mg daily, such as once daily, and said MTX is administered at a dosage of 5 to 30 mg once weekly.

In some embodiments, the DAS28 score is reduced to below 3.2 by the treatment of the present disclosure (low disease activity). In some embodiments, the DAS28 score is reduced to below 2.6 by the treatment of the present disclosure (remission).

### CDAI Score

The CDAI (Clinical Disease Activity Index) is a useful clinical composite score for following patients with rheumatoid arthritis. The CDAI is the sum of 4 outcome parameters: tender and swollen joint counts (28 joints assessed) and patient's and physician's global assessments of disease activity (on a 0-10-cm visual analog scale). The CDAI is the same as the Simplified Disease Activity Index (SDAI), except that the SDAI includes the C-reactive protein level.

Descriptive changes in CDAI where CDAI=SJC (28)+TJC (28)+PGA+IGA;
SJC (28): Swollen 28-Joint Count (shoulders, elbows, wrists, MCPs, PIPs including thumb IP, knees);
TJC (28): Tender 28-Joint Count (shoulders, elbows, wrists, MCPs, PIPs including thumb IP, knees);
PGA: Patient Global Disease Activity (patient's self-assessment of overall RA disease activity on a scale 0-100 where 100 is maximal activity);
IGA: Physician's Global Disease Activity (evaluator's assessment of the subject's overall RA disease activity on a scale 0-100 where 100 is maximal activity)

Remission is considered achieved if the score is between 0 and <2.8; Low disease activity corresponds to 2.8 to <10. Moderate disease activity is between 10 and ≤22, while high disease activity is strictly above 22 (CDAI>22).

In some embodiments the present disclosure provides a compound of formula (I) as defined herein in combination with MTX for use in the treatment of rheumatoid arthritis in a subject with a CDAI>22,
wherein said compound is administered at a dosage of more than 41.5 mg daily, such as at least about 50 mg daily, such as at least about 62 mg daily, such as at least about 75 mg daily, such as once daily, and said MTX is administered at a dosage of 5 to 30 mg once weekly.

In some embodiments said subject's CDAI score during and/or after treatment is reduced to between 10 and ≤22 (moderate activity), such as reduced to between 2.8 to <10 (low activity), such as reduced to between 0 and <2.8 (remission).

In some embodiments the present disclosure provides a compound of formula (I) as defined herein in combination with MTX for use in the treatment of rheumatoid arthritis in a subject with a CDAI score of between 10 and ≤22,
wherein said compound is administered at a dosage of more than 41.5 mg daily, such as at least about 50 mg daily, such as at least about 62 mg daily, such as at least about 75 mg daily, such as once daily, and said MTX is administered at a dosage of 5 to 30 mg once weekly.

In some embodiments the present disclosure provides a compound of formula (I) as defined herein in combination with MTX for use in the treatment of rheumatoid arthritis in a subject with a CDAI score of between 2.8 to <10, wherein said compound is administered at a dosage of more than 41.5 mg daily, such as at least about 50 mg daily, such as at least about 62 mg daily, such as at least about 75 mg daily, such as once daily, and said MTX is administered at a dosage of 5 to 30 mg once weekly.

Reference to a decrease in a subject's CDAI score and reference to a reduction in a subject's CDAI score is used interchangeably herein.

In some embodiments said subject's CDAI score during and/or after treatment result in a 5-point decrease, such as a 10-point decrease, such as a 15-point decrease in the subjects CDAI.

In some embodiments, the CDAI score is reduced by the treatment of the present disclosure by at least 2 points, such as at least 3 points, such as at least 4 points, such as at least 5 points, such as at least 6 points, such as at least 7 points, such as at least 8 points, such as at least 9 points, such as at least 10 points, such as at least 11 points, such as at least 12 points, such as at least 13 points, such as at least 14 points, such as at least 15 points, such as at least 16 points, such as at least 17 points, such as at least 18 points, such as at least 19 points, such as at least 20 points.

In some embodiments, the CDAI score is reduced to below 10 by the treatment of the present disclosure (low disease activity). In some embodiments, the DAS28 score is reduced to below 2.8 by the treatment of the present disclosure (remission).

In some embodiments, the subjects CDAI score is reduced by the treatment of the present disclosure by 5 points or more, such as 10 points or more, such as 15 point or more.

The decrease in the subjects CDAI may be indicated at any time during treatment. In some embodiments the decrease in the subjects CDAI is indicated at week 2 after treatment initiation. In some embodiments the decrease in the subjects CDAI is indicated at week 4 after treatment initiation. In some embodiments the decrease in the subjects CDAI is indicated at end of treatment period. In some embodiments the decrease in the subjects CDAI is indicated at week 5 after treatment initiation. In some embodiments the decrease in the subjects CDAI is indicated at one week follow-up after last dosage.

### ACR-Score

The ACR (American College of Rheumatology) Criteria is a standard criterion to measure the effectiveness of various arthritis medications or treatments in clinical trials for RA.

The ACR response rates ACR20, ACR50, and ACR70 are defined as ≥20%, ≥50% and ≥70% improvement, respectively, in swollen and tender joint counts (SJC/TJC) and 3 of the following 5 assessments: Patient's Global Assessment of Disease Activity, Physician's Global Assessment of Disease Activity, Patient's Assessment of Pain, Health Assessment Questionnaire (HAQ-DI), and C-Reactive Protein (CRP).

In some embodiments, the ACR-score is improved by the treatment of the present disclosure. In some embodiments, the present therapy result in a ≥20%, a ≥50% or ≥70% improvement in ACR response rates.

### Pharmaceutical Composition

The compounds for use according to the present disclosure, including a compound of formula (I) including tautomeric and stereoisomeric forms thereof, or a pharmaceutically acceptable derivative thereof, and methotrexate (MTX), may be provided in any suitable formulation.

### MTX

In one embodiment of the present disclosure MTX is formulated for oral administration, such as in the form of tablets, capsules or oral solutions or suspensions.

In one embodiment of the present disclosure MTX is formulated as a liquid, such as a liquid suitable for intravenous administration or injection, such as a liquid suitable for subcutaneous injection.

In one embodiment of the present disclosure MTX is formulated for extended release.

In one embodiment of the present disclosure MTX is formulated for immediate release.

### AP1189

It is also an aspect of the present disclosure to provide a pharmaceutical composition comprising (E)-N-[1-(2-nitrophenyl)-1-H-pyrrole-2-yl-allylideneamino]-guanidine (compound of formula I), including tautomeric and stereoisomeric forms thereof, and pharmaceutically acceptable salts thereof, for the medical uses disclosed herein.

In one embodiment of the present disclosure the compound of formula (I) including tautomeric and stereoisomeric forms thereof, and pharmaceutically acceptable salts thereof, is formulated for oral administration, such as in the form of tablets, capsules, or oral solutions or suspensions.

In one embodiment of the present disclosure the compound of formula (I) including tautomeric and stereoisomeric forms thereof, and pharmaceutically acceptable salts thereof, is formulated as a liquid, such as a liquid for intravenous administration or continuous infusion, or a liquid for injection.

In one embodiment of the present disclosure the compound of formula (I) including tautomeric and stereoisomeric forms thereof, and pharmaceutically acceptable salts thereof, is formulated for extended release.

In one embodiment of the present disclosure the compound of formula (I) including tautomeric and stereoisomeric forms thereof, and pharmaceutically acceptable salts thereof, is formulated for immediate release.

In a particular embodiment the compound of formula (I) including tautomeric and stereoisomeric forms thereof, and pharmaceutically acceptable salts thereof, is formulated for oral administration.

In a particular embodiment the compound of formula (I) is {3-[1-(2-nitrophenl-1H-pyrrol-2-yl]-allylidene}-aminoguanidine, for example (E)-N-trans-{3-[1-(2-nitrophenyl)-1H-pyrrol-2-yl]-allylidene}-aminoguanidine, or a pharmaceutically acceptable salt thereof, such as (E)-N-trans-{3-[1-(2-nitrophenyl)-1H-pyll-2-yl]-allylidene}-aminoguanidinium acetate (AP1189) or (E)-N-trans-{3-[1-(2-nitrophenyl)-1H-pyrrol-2-yl]-allylidene}-aminoguanidinium succinate, wherein said compound is formulated for oral administration.

In one embodiment said compound is formulated as an solid oral dosage form, such as a tablet.

In one embodiment said compound is formulated as a powder, such as an oral powder, such as an oral powder suitable for suspension in a liquid.

In one embodiment said compound is formulated as a suspension comprising dissolved oral powder.

In one embodiment said compound is formulated as an oral suspension, such as a suspension for oral administration.

To circumvent degradation at low pH of the gastric compartment, AP1189 has been formulated as an enteric coated tablet and later as an alkaline suspension. It has recently been demonstrated that a subset of the pharmaceutically acceptable salts of AP1189, including AP1189 in its acetate salt and succinate salt forms, are highly soluble at low pH and hence do not require protection from low pH, allowing the use also of solid oral formulations targeting the gastric compartment such as immediate release solid oral formulations (PCT/EP2022/066906).

In one embodiment there is provided a unit dosage form comprising a compound of formula (I) including tautomeric and stereoisomeric forms thereof, and pharmaceutically acceptable salts thereof, for the uses disclosed herein.

In one embodiment there is provided an oral formulation comprising a compound of formula (I) including tautomeric and stereoisomeric forms thereof, and pharmaceutically acceptable salts thereof, for the uses disclosed herein.

In one embodiment there is provided an oral formulation comprising a compound selected from the group consisting of:
(E)-N-[1-(2-nitrophenyl)-1-H-pyrrole-2-yl-allylideneamino]-guanidinium acetate, including tautomeric and stereoisomeric forms thereof;
(E)-N-[1-(2-nitrophenyl)-1-H-pyrrole-2-yl-allylideneamino]-guanidinium succinate including tautomeric and stereoisomeric forms thereof;
(E)-N-[1-(2-nitrophenyl)-1-H-pyrrole-2-yl-allylideneamino]-guanidine DL-mandelic acid salt including tautomeric and stereoisomeric forms thereof;
(E)-N-[1-(2-nitrophenyl)-1-H-pyrrole-2-yl-allylideneamino]-guanidine hippuric acid salt including tautomeric and stereoisomeric forms thereof;
(E)-N-[1-(2-nitrophenyl)-1-H-pyrrole-2-yl-allylideneamino]-guanidine L-lactic acid salt including tautomeric and stereoisomeric forms thereof;
(E)-N-[1-(2-nitrophenyl)-1-H-pyrrole-2-yl-allylideneamino]-guanidinium besylate including tautomeric and stereoisomeric forms thereof;
(E)-N-[1-(2-nitrophenyl)-1-H-pyrrole-2-yl-allylideneamino]-guanidinium oxoglutarate including tautomeric and stereoisomeric forms thereof;
(E)-N-[1-(2-nitrophenyl)-1-H-pyrrole-2-yl-allylideneamino]-guanidine formic acid salt including tautomeric and stereoisomeric forms thereof;
(E)-N-[1-(2-nitrophenyl)-1-H-pyrrole-2-yl-allylideneamino]-guanidine DL-lactic acid salt including tautomeric and stereoisomeric forms thereof;
(E)-N-[1-(2-nitrophenyl)-1-H-pyrrole-2-yl-allylideneamino]-guanidine glutaric acid salt including tautomeric and stereoisomeric forms thereof;
(E)-N-[1-(2-nitrophenyl)-1-H-pyrrole-2-yl-allylideneamino]-guanidine adipic acid salt including tautomeric and stereoisomeric forms thereof; and
(E)-N-[1-(2-nitrophenyl)-1-H-pyrrole-2-yl-allylideneamino]-guanidinium nitrate salt including tautomeric and stereoisomeric forms thereof.

In some embodiments, the pharmaceutically acceptable salt of AP1189 according to the present disclosure is a crystalline or polymorphic form of a pharmaceutically acceptable salt of AP1189 selected from the group consisting of: AP1189 acetate, AP1189 succinate, the DL-mandelic acid salt of AP1189, the hippuric acid salt of AP1189, the L-lactic acid salt of AP1189, the besylate salt of AP1189, the oxoglutarate salt of AP1189, the formic acid salt of AP1189, the DL-lactic acid salt of AP1189, the glutaric acid salt of AP1189, the adipic acid salt of AP1189 and the nitrate salt of AP1189. Polymorphic forms are prepared and disclosed in PCT/EP2022/066884, the disclosure of which is incorporated by reference herewith.

In some embodiments there is provided an oral formulation comprising a compound of formula (I) including tautomeric and stereoisomeric forms thereof, and pharmaceutically acceptable salts thereof, and comprising at least one pharmaceutically acceptable excipient, for the uses disclosed herein.

In some embodiments said oral formulation delivers or releases said compound to the gastric compartment (or stomach), such as primarily or predominantly delivers or releases said compound to the gastric compartment (or stomach).

In some embodiments said oral formulation delivers or releases said compound in the gastric compartment by immediate release, by delayed release, by burst release, or by any means of releasing said compound primarily or predominantly in the gastric compartment.

In some embodiments said oral formulation delivers or releases not less than about 65% to about 80% of said compound in the gastric compartment; such as not less than about 65% of said compound, such as not less than about 70%, such as not less than about 75%, such as not less than about 80%, such as not less than about 85%, such as not less than about 90%, such as not less than about 95% of said compound in the gastric compartment.

In some embodiments said oral formulation immediately releases said compound in the gastric compartment.

In some embodiments said oral formulation releases said compound in the gastric compartment for gastric absorption of said compound.

In some embodiments said oral formulation releases said compound in the gastric compartment for absorption of said compound over the gastric mucus layer.

In some embodiments said oral formulation is designed for gastric delivery.

In some embodiments said oral formulation is designed for gastric release.

In some embodiments said oral formulation is designed for gastric absorption.

In some embodiments said oral formulation is a solid oral formulation.

In some embodiments said solid oral formulation is a solid oral dosage form.

In some embodiments said solid oral dosage form is an immediate release solid oral dosage form.

In some embodiments said oral formulation is a tablet. In some embodiments said solid oral formulation is a tablet. In some embodiments said solid oral dosage form is a tablet.

In some embodiments there is provided a solid oral formulation, solid oral dosage form or tablet, comprising a compound of formula (I) as disclosed herein, wherein not less than about 65% to about 80% of said compound is dissolved into solution in the gastric compartment.

In some embodiments there is provided a solid oral formulation, solid oral dosage form or tablet, comprising a compound of formula (I) as disclosed herein, wherein not less than about 65%, such as not less than about 70%, such as not less than about 75%, such as not less than about 80%, such as not less than about 85%, such as not less than about 90%, such as not less than about 95% of said compound is dissolved into solution in the gastric compartment.

In some embodiments said oral formulation is a delayed release tablet comprising a compound of formula (I) as disclosed herein targeting release in the gastric compartment.

In some embodiments said oral formulation is a gastric retentive delayed-release formulation. In some embodiments said oral formulation is a gastroretentive tablet. In some embodiments said gastroretentive tablet is a gastroretentive double-layered tablet formulation. In some embodiments said gastroretentive double-layered tablet formulation is a gastric swelling system (GSS), such as a GSS consisting of a swelling layer and a drug release layer.

In one embodiment the immediate release solid oral formulation, such as immediate release solid oral dosage form, such as immediate release tablet of the present disclosure comprises a pharmaceutically acceptable salt of AP1189 selected from the group consisting of:
(E)-N-[1-(2-nitrophenyl)-1-H-pyrrole-2-yl-allylideneamino]-guanidinium acetate, including tautomeric and stereoisomeric forms thereof;
(E)-N-[1-(2-nitrophenyl)-1-H-pyrrole-2-yl-allylideneamino]-guanidinium succinate including tautomeric and stereoisomeric forms thereof;
(E)-N-[1-(2-nitrophenyl)-1-H-pyrrole-2-yl-allylideneamino]-guanidine DL-mandelic acid salt including tautomeric and stereoisomeric forms thereof;
(E)-N-[1-(2-nitrophenyl)-1-H-pyrrole-2-yl-allylideneamino]-guanidine hippuric acid salt including tautomeric and stereoisomeric forms thereof;
(E)-N-[1-(2-nitrophenyl)-1-H-pyrrole-2-yl-allylideneamino]-guanidine L-lactic acid salt including tautomeric and stereoisomeric forms thereof;
(E)-N-[1-(2-nitrophenyl)-1-H-pyrrole-2-yl-allylideneamino]-guanidinium besylate including tautomeric and stereoisomeric forms thereof;
(E)-N-[1-(2-nitrophenyl)-1-H-pyrrole-2-yl-allylideneamino]-guanidinium oxoglutarate including tautomeric and stereoisomeric forms thereof;
(E)-N-[1-(2-nitrophenyl)-1-H-pyrrole-2-yl-allylideneamino]-guanidine formic acid salt including tautomeric and stereoisomeric forms thereof;
(E)-N-[1-(2-nitrophenyl)-1-H-pyrrole-2-yl-allylideneamino]-guanidine DL-lactic acid salt including tautomeric and stereoisomeric forms thereof;
(E)-N-[1-(2-nitrophenyl)-1-H-pyrrole-2-yl-allylideneamino]-guanidine glutaric acid salt including tautomeric and stereoisomeric forms thereof;
(E)-N-[1-(2-nitrophenyl)-1-H-pyrrole-2-yl-allylideneamino]-guanidine adipic acid salt including tautomeric and stereoisomeric forms thereof; and
(E)-N-[1-(2-nitrophenyl)-1-H-pyrrole-2-yl-allylideneamino]-guanidinium nitrate salt including tautomeric and stereoisomeric forms thereof, and at least one pharmaceutically acceptable excipient.

In one embodiment the immediate release solid oral formulation, such as immediate release solid oral dosage form, such as immediate release tablet of the present disclosure comprises
(E)-N-[1-(2-nitrophenyl)-1-H-pyrrole-2-yl-allylideneamino]-guanidinium acetate, including tautomeric and stereoisomeric forms thereof, or
(E)-N-[1-(2-nitrophenyl)-1-H-pyrrole-2-yl-allylideneamino]-guanidinium succinate, including tautomeric and stereoisomeric forms thereof,
   and at least one pharmaceutically acceptable excipient.

In some embodiments there is provided an oral formulation, such as a solid oral formulation, such as an immediate-release solid oral formulation, comprising a compound of formula (I) as disclosed herein, wherein not less than about 65% to about 80% of said compound is dissolved into solution in about 5 minutes.

In some embodiments there is provided an oral formulation, such as a solid oral formulation, such as an immediate-release solid oral formulation, comprising a compound of formula (I) as disclosed herein, wherein not less than about 65% to about 80% of said compound is dissolved into solution in about 5 minutes at a pH of about 1 to 3, such as a pH of about 0.5 to 3.5; such as a pH of about 0.5 to 1, such as a pH of about 1 to 1.5, such as a pH of about 1.5 to 2, such as a pH of about 2 to 2.5, such as a pH of about 2.5 to 3, such as a pH of about 3 to 3.5.

In some embodiments there is provided an oral formulation, such as a solid oral formulation, such as an immediate-release solid oral formulation, comprising a compound of formula (I) as disclosed herein, wherein not less than about 65% to about 80% of said compound is dissolved into solution in about 5 minutes at a pH of about 1.2.

In some embodiments there is provided an oral formulation, such as a solid oral formulation, such as an immediate-release solid oral formulation, comprising a compound of formula (I) as disclosed herein, wherein not less than about 65% to about 80% of the of the nominal dose of said compound is released in about 5 minutes in a monograph dissolution test.

In some embodiments there is provided an oral formulation, such as a solid oral formulation, such as an immediate-release solid oral formulation, comprising a compound of formula (I) as disclosed herein, wherein not less than about 80% of said compound is dissolved into solution in about 10 minutes, such as at a pH of about 1.2.

In some embodiments there is provided an oral formulation, such as a solid oral formulation, such as an immediate-release solid oral formulation, comprising a compound of formula (I) as disclosed herein, wherein the disintegration time of said oral formulation such as solid oral formulation is from ½ minute to 10 minutes, such as ½ minute to 1 minute, such as 1 to 2 minutes, such as 2 to 3 minutes, such as 3 to 4 minutes, such as 4 to 5 minutes, such as 5 to 6 minutes, such as 6 to 7 minutes, such as 7 to 8 minutes, such as 8 to 9 minutes, such as 9 to 10 minutes.

In some embodiments the solid oral dosage form comprises a compound of formula (I) as defined herein at a dosage from about 25 mg to about 650 mg per dosage form, such as about 25 mg, such as about 50 mg, such as about 100 mg, such as about 150 mg, such as about 200 mg, such as about 250 mg, such as about 300 mg, such as about 350 mg, such as about 400 mg, such as about 450 mg, such as about 500 mg, such as about 550 mg, such as about 600 mg, such as about 650 mg compound (calculated as the free base).

### EXAMPLES

A double-blind, multi-center, two-part, randomized, placebo-controlled study of the safety, tolerability, and efficacy of 4 weeks of treatment with AP1189 in early rheumatoid arthritis (RA) patients with active joint disease (Clinical phase 11a). The study population will consist of newly diagnosed subjects with severe active RA (CDAI (Clinical disease activity score)>22) who are to start up-titration with methotrexate (MTX).

### Example 1

Doses of 50 mg and 100 mg AP1189 are selected. The doses used in this Example are calculated based on the specific acetate salt form. Hence, a dosage of “100 mg AP1189” (acetate salt) as referred to herein correspond to a dosage of 83 mg AP1189 free base.

The peak respectively trough concentrations identified in the repeated dose part of the Phase I study with AP1189 were during steady conditions as follows:

| | C Max | C Trough |
|---|---|---|
| | (mean +/− SD, | (Mean +/− SD, |
| | N = 9) | N = 9) |
| 50 mg once daily | 180 ± 38 ng/ml | 46 ± 14 ng/ml |
| 100 mg once daily | 427 ± 76 ng/ml | 100 ± 25 ng/ml |
| 200 mg once daily | 893 ± 198 ng/ml | 186 ± 45 ng/ml |

### Primary Safety Endpoint

The safety of AP1189 against placebo by evaluating adverse events (AEs), serious adverse events (SAEs), and laboratory abnormalities.

### Primary Efficacy Endpoint

The change in CDAI after 4 weeks of treatment compared to baseline will be evaluated by assessing the following, by treatment group:
Mean change in CDAI from baseline
Proportion of subjects with a change in CDAI score from severe (CDAI>22) to moderate (CDAI≤22) at week 4 compared to baseline.

### Secondary Efficacy Endpoints

The effects of AP1189 against placebo will be evaluated by assessing the following by treatment group:
Proportion of subjects achieving a reduction of more than 10 (ten) swollen and/or tender joints (SJC and TJC, summarized) at week 4 compared to baseline
Proportion of subjects achieving a change in CDAI score at week 4 compared to baseline
   Proportion of subjects with a 5-point decrease
   Proportion of subjects with a 10-point decrease
   Proportion of subjects with a 15-point decrease
Proportion of subjects achieving a change in DAS28 from DAS28>3.2 to DAS28≤3.2 at week 4 compared to baseline
Change of HAQ-DI at week 4 compared to baseline
Change of FACIT-Fatigue at week 4 compared to baseline
Proportion of subjects achieving ACR response assessed by ACR 20, ACR 50, and AC70

### Tertiary Efficacy Endpoints

The effect of AP1189 compared to placebo at week 4 compared to baseline will be further evaluated by assessing the following by treatment group:
CXCL13, IL-1β, IL-6, IL-10, and TNF-α.
Synovial biopsy at baseline and after 4 weeks treatment (only Part 2 at selected sites).

### Study Design

This study is a multicenter, two-part, randomized, double-blind, placebo-controlled, 4-week study with repeated doses of AP1189. The study population will consist of newly diagnosed subjects with severe active RA (CDAI>22) who are to start up-titration with MTX. A minimum of 90 subjects are expected to complete the study; plus 45 subjects from Bulgaria and/or Moldova for a sub-study. Up to 120 subjects are planned to be enrolled to account for discontinuation rate, and up to 60 subjects for the sub-study.

Subjects who fulfill the enrollment criteria will be randomized in a 2:1 ratio in group A and B. One group will receive active treatment, and the other group will receive a placebo. Group C/D will have the same 2:1 ratio between active and placebo.
Group A (12 subjects): AP1189 dose 50 mg, once daily for 4 weeks (28 days) plus MTX (10-25 mg) weekly
Group B (6 subjects): placebo for 4 weeks (28 days) plus MTX (10-25 mg) weekly
Group C (12 subjects): AP1189 dose 100 mg, once daily for 4 weeks (28 days) plus MTX (10-25 mg) weekly
Group D (6 subjects): placebo for 4 weeks (28 days) plus MTX (10-25 mg) weekly

### Number of Subjects Part 1

A minimum of 24 subjects is expected to complete Part 1 of the study. About 32 subjects are planned to be enrolled in accounting for discontinuation rate.

### Part 2

All subjects will be randomized into one design only, either design 1, 2, or 3
Design 1: AP1189 dose 50 mg (min. 44 subjects) or placebo (min. 22 subjects), once daily for 4 weeks (28 days) plus MTX (10-25 mg) weekly
Design 2: AP1189 dose 100 mg (min. 44 subjects) or placebo (min. 22 subjects), once daily for 4 weeks (28 days) plus MTX (10-25 mg) weekly.
Design 3: Continue with the same doses as in Part 1, in a 1:1:1 ratio (AP1189 50 mg (min. 22 subjects), AP1189 100 mg (min. 22 subjects) or placebo (min. 22 subjects) plus MTX (10-25 mg) weekly

### Number of Subjects in Part 2

A minimum of 66 subjects is expected to complete Part 2 of the study, and 45 subjects in the sub-study. About 88 subjects are planned to be enrolled in the main study accounting for discontinuation rate, and about 60 subjects in the sub-study.

### Study Duration

Total study duration is 18 months, and the study duration for each subject is approximately and up to 10 weeks.

### Number of Investigational Sites

The study is to be conducted at sites in Europe, and Moldova.

### Study Population

The study population will consist of subjects with severe active RA, defined as CDAI>22, who are about to begin up-titration with MTX.

### Inclusion Criteria

1. Written informed consent has been obtained prior to initiating any study specific procedures
2. Male and female subjects, 18 to 85 years of age
3. Confirmed diagnosis of RA according to the 2010 ACR/EULAR RA classification criteria
4. Arthritis with joint swelling and tenderness of a minimum of three joints out of 68 joints tested
5. Candidate for MTX treatment
6. Is about to begin treatment with MTX
7. Tested positive for anti-CCP or RF
8. Severe active RA (CDAI>22) at screening and baseline
9. Negative QFG-IT (Mantoux test can be used if QFG-IT is not possible)
10. Subjects should be able to complete (read and write) the PRO questionnaires
11. Females of child-bearing potential may only participate if using reliable means of contraception or are post-menopausal (menstrual periods stopped at least 12 months ahead of the enrolment in the trial). Surgically sterilized women at least 6 months prior to screening
12. Females of childbearing potential must have a negative pregnancy test at screening and baseline.

### Exclusion Criteria

Subjects meeting any of the following criteria are not eligible for participation in the study:
1. Participation in any other study involving investigational drug(s) within 4 weeks prior to study entry
2. Major surgery (including joint operation) within 8 weeks prior to screening or planned surgery within 1 month following randomization
3. Rheumatic autoimmune disease other than RA, including SLE, MCTD, scleroderma, polymyositis, or significant systemic involvement secondary to RA (e.g., vasculitis, pulmonary fibrosis or Felty's syndrome). Sjögren syndrome with RA is allowable
4. Functional class IV as defined by the ACR Criteria for Classification of Functional Status in RA or wheelchair/bedbound
5. Prior history of or current inflammatory joint disease other than RA (e.g., gout, reactive arthritis, psoriatic arthritis, seronegative spondyloarthropathy, Lyme disease)
6. Subjects with fibromyalgia
7. Initiation or change in dose for NSAIDs (including low-dose aspirin and COX-2 inhibitors) within 2 weeks prior to dosing with AP1189
8. Corticosteroids are prohibited within 2 weeks prior to screening (and during the entire treatment period and until the final visit)
9. Evidence of serious uncontrolled concomitant cardiovascular, nervous system, pulmonary (including obstructive pulmonary disease), renal, hepatic, endocrine (including uncontrolled diabetes mellitus), or gastrointestinal disease
10. Have prior renal transplant, current renal dialysis or severe renal insufficiency (determined by a derived glomerular filtration rate (GFR) using Cockcroft Gault formula of ≤30 ml/min/1.73 m² calculated by the local lab)
11. Uncontrolled disease states, such as asthma, psoriasis, or inflammatory bowel disease where flares are commonly treated with oral or parenteral corticosteroids
12. Evidence of active malignant disease (except basal cell carcinoma of the skin that has been excised and cured)
13. Pregnant women or nursing (breastfeeding) mothers
14. History of alcohol, drug, or chemical abuse within the 6 months prior to screening
15. Neuropathies or other painful conditions that might interfere with pain evaluation
16. Body weight of >150 kg.

Exclusion criteria 17-20 only apply to subjects in Norway:
17. Evidence of moderate and/or severe organ dysfunction
18. Abnormal chest x-ray (as per the discretion of the investigator)
19. Evidence of positive hepatitis serology
20. Evidence of peptic ulcer disease

The study drug information is presented in the below tables.

TABLE 1
Test Treatment

| | |
|---|---|
| Name | AP1189 |
| Dosage strength | 50 mg, 100 mg |
| Formulation | Powder (for suspension) |
| Route of | Oral |
| Supplier | SynAct Pharma |

TABLE 2
Reference Treatment

| | |
|---|---|
| Name | Placebo |
| Dosage strength | 0 mg |
| Formulation | Powder |
| Route of | oral |
| Supplier | SynAct Pharma |

### Description of AP1189 Product:

### IUPAC: E-N-[trans-3-{1-(2-nitrophenyl)-1H-pyrrole-2-yl}-allylideneamino]guanidinium acetate

### Priorietary: AP1189

The substance is an acetic acid salt that appears as an odorless, yellow to brownish solid. The molecular weight is 358.35 for the acetate salt and 298.30 for the free base.

### Methotrexate (MTX)

All subjects will follow the local guideline for starting treatment with MTX and continue MTX treatment throughout the study.

### MTX Treatment Instruction in Case of Elevation in Liver Enzymes

It is recommended with more frequent blood test in case of elevation of liver enzymes.

Transaminase Increase:

| Laboratory Value | Action |
|---|---|
| ALT and/or AST | IMP and MTX remains unchanged. |
| increases up to >1 | It is recommended with more |
| to ≤3 × Upper Limit | frequent blood test in case of |
| of Normal (ULN) | elevation of liver enzymes. |
| And bilirubin is | |
| within the normal | |
| range | |
| ALT and/or AST | Pause IMP dosing until ALT and/or AST <3 × ULN and |
| increases >3 | follow recommendations above for >1 to <3 × ULN |
| to ≤5 × ULN | Upon normalization of ALT and/or AST, IMP resumes |
| And bilirubin is | For persistent increases >3 × ULN, discontinue IMP |
| within the normal | |
| range | |
| ALT and/or | MTX and IMP discontinue |
| AST >5 × ULN | |
| Bilirubin > | MTX and IMP treatment |
| normal range | discontinue |

IMP—Investigational medicinal product (here AP1189, acetate salt)

### Folic Acid

It is possible that AEs commonly associated with MTX treatment will occur. To minimize MTX toxicity, all subjects treated with MTX should be on folic acid or equivalent at a dose of at least 5 mg/week according to local guidelines and at the discretion of the investigator. Folic acid can either be given as a single dose weekly or be divided into daily doses to achieve at least 5 mg folic acid per week.

### Laboratory Assessments

The latest updated reference ranges from the local laboratory will be used to identify subjects with clinically notable laboratory values.

### Hematology

Hemoglobin, white blood cell (WBC) count (total and differential: leukocytes, neutrophils, eosinophils, basophils, lymphocytes, monocytes), red blood cells (RBC), thrombocytes and hemoglobin A1c (HbA1C). The hematology blood samples will be taken at screening, baseline, after 2 weeks and 4 weeks treatment.

### Biochemistry

Sodium, potassium, chloride, calcium, glucose, creatinine, urea, albumin, unconjugated and total bilirubin, aspartate transaminase (AST), alanine transaminase (ALT), alkaline phosphatase (ALP), gamma-glutamyl transferase (GGT) and INR. The biochemistry blood samples will be taken at all visits. A serum β-HCG pregnancy test will be taken at screening.

### Thyroid Function

Thyroxine (T4) free, triiodothyronine (T3) total or free, and the thyroid-stimulating hormone (TSH). Blood samples for measuring the thyroid function.

### Urinalysis

A dipstick urine test for blood, protein, and glucose will be performed at the site at the Screening Visit. A urine sample may be sent for urine culture.

### Serology

RF or anti-CCP, HBsAg, HBV antibody and HCV antibody.

RF is an antibody that is detectable in the blood of approximately 80% of adults with RA.

CRP is an acute phase reactant, a protein made by the liver and released into the blood within a few hours after tissue injury, the start of an infection, or other cause of inflammation. The CRP will most often be increased by inflammation. One of the aims of treatment is to reduce the CRP to normal levels. CRP will be measured at screening, baseline, after 2 weeks and 4 weeks treatment.

### Safety (AE and SAE)

Safety measures (AEs, SAEs, including laboratory abnormalities) will be registered during the whole study duration.

Safety Assessments (Sub-study, only Part 2)

The arthroscopy sub-study in Part 2 (only selected sites), will assess the effect of 4 weeks treatment with AP1189/placebo compared to baseline by examining synovial fluid: (evaluating the change in the percentage of polymorphs, monocytes, and lymphocytes in synovial fluid).

In RA the immunohistological features of synovial inflammation change as the clinical manifestations change in response to conventional disease-modifying antirheumatic drugs, pulse methylprednisolone, or intra-articular glucocorticoids.

### Efficacy Assessments

### Swollen Joint Count (SJC) and Tender Joint Count (TJC)

An assessment of 66 joints for swelling and 68 joints for tenderness will be made at screening, baseline, after 2 weeks and 4 weeks treatment. Joints will be assessed and classified as swollen/not swollen and tender/not tender by pressure and joint manipulation on physical examination. The subject will be asked for pain sensations on these manipulations and watched for spontaneous pain reactions. Any positive response to pressure, movement, or both will then be categorized as tender-versus-nontender. Swelling is defined as palpable fluctuating synovitis of the joint. Swelling secondary to osteoarthrosis will be assessed as not swollen unless there is unmistakable fluctuation.

Joint assessments of one particular subject should be performed by the same assessor (if at all possible) throughout the trial to minimize inter-observer variation.

### Clinical Disease Activity Index (CDAI)

The CDAI is a clinical composite score for following patients with RA.

Descriptive changes in CDAI where CDAI=SJC (28)+TJC (28)+PGA+IGA;
SJC (28): Swollen 28-Joint Count (shoulders, elbows, wrists, MCPs, PIPs including thumb IP, knees);
TJC (28): Tender 28-Joint Count (shoulders, elbows, wrists, MCPs, PIPs including thumb IP, knees);
PGA: Patient Global Disease Activity (patient's self-assessment of overall RA disease activity on a scale 0-100 where 100 is maximal activity);
IGA: Physician's Global Disease Activity (evaluator's assessment of the subject's overall RA disease activity on a scale 0-100 where 100 is maximal activity)

The CDAI will be scored at screening, baseline, after 2 weeks and 4 weeks treatment, and at the final visit (one week after final dose).

### Disease Activity Score 28 (DAS28)

The DAS28 is a combined index for measuring disease activity in RA. The index includes swollen and tender joint counts, CRP, and general health status. In this trial CRP will be used to calculate the DAS28 score. The index is calculated using the following formula:

DAS28−CRP(4)=0.56*sqrt(TJC28)+0.28*sqrt(SJC28)+0.36*ln(CRP+1)+0.014*GH+0.96

Where, TJC=tender joint count on 28 joints, SJC=swollen joint count on 28 joints, ln=natural log, CRP=C-reactive Protein, and GH=general health, i.e., patient's global assessment of disease activity (100-mm VAS).

The DAS28 provides an absolute indication of RA disease activity on a scale of 0.49 to 9.07
A DAS28 value>5.1 corresponds to a high disease activity
A DAS28 value between 3.2 and 5.1 corresponds to a moderate disease activity
A DAS28 value between 2.6 and 3.2 corresponds to a low disease activity
A DAS28 value<2.6 corresponds to remission

Compared to an initial value the disease activity of the subject can be classified as follows:

| | | DAS28 decrease from initial value | | |
|---|---|---|---|---|
| Current DAS28 | | >1.2 | >0.6 but ≤1.2 | ≤0.6 |
| ≤3.2 | Inactive | Good | Moderate | No |
| | | improvement | improvement | improvement |
| >3.2 | Moderate | Moderate | Moderate | No |
| but ≤5.1 | | improvement | improvement | improvement |
| >5.1 | Very | Moderate | No | No |
| | active | improvement | improvement | improvement |

The DAS28 will be scored at baseline, after 2 weeks and 4 weeks treatment, and at the final visit.

### Physician's Global Assessment of Disease Activity VAS (“Investigator Global VAS”)

The physician's assessment of the subject's current disease activity on a 100 mm horizontal VAS. The extreme left end of the line should be described as “no disease activity” (symptom-free and no arthritis symptoms) and the extreme right end as “maximum disease activity.” The efficacy assessor should complete this. Investigator Global VAS will be measured at screening, baseline, after 2 weeks and 4 weeks treatment.

### Patient's Global Assessment of Disease Activity VAS (“Patient Global VAS”)

The subject's overall assessment of their current disease activity on a 100 mm horizontal VAS. The extreme left end of the line should be described as “no disease activity” symptom-free and no arthritis symptoms) and the extreme right end as “maximum disease activity” (maximum arthritis disease activity). Patient Global VAS will be measures at screening, baseline, after 2 weeks and 4 weeks treatment.

### Patient's Assessment of Pain VAS (“Patient Pain VAS”)

The subject's assessment of his/her current level of pain on a 100 mm horizontal VAS. The extreme left end of the line should be described as “no pain” and the extreme right end as “unbearable pain.” Patient Pain VAS will be measured at screening, baseline, after 2 weeks and 4 weeks treatment.

### Quality of Life and Physical Function

### Functional Assessment of Chronic Illness Therapy-Fatigue (FACIT-Fatigue)

The FACIT-Fatigue assessment is a 13-item questionnaire with subjects scoring each item on a 5-point scale. The assessment was originally developed for chronic illnesses and is now validated for patients with RA. FACIT-Fatigue will be scored at baseline, after 2 weeks and 4 weeks treatment.

### Health Assessment Questionnaire-Disability Index (HAQ-DI)

HAQ-DI is a validated tool to evaluate physical function. It consists of 20 questions referring to 8 component sets: dressing/grooming, arising, eating, walking, hygiene, reach, grip, and activities. HAQ-DI will be scored at baseline, after 2 weeks and 4 weeks treatment.

### American College of Rheumatology Response Rates

The ACR (American College of Rheumatology) Criteria is a standard criterion to measure the effectiveness of various arthritis medications or treatments in clinical trials for RA.

The ACR response rates ACR20, ACR50, and ACR70 are defined as ≥20%, ≥50% and ≥70% improvement, respectively, in swollen and tender joint counts (SJC/TJC) and 3 of the following 5 assessments: Patient's Global Assessment of Disease Activity (see above), Physician's Global Assessment of Disease Activity (see above), Patient's Assessment of Pain (see above), Health Assessment Questionnaire (HAQ-DI, see above), and C-Reactive Protein (CRP).

### Pharmacokinetic Assessments

### PK Sample Collection

Plasma PK samples for exposure-response analysis will be taken after 1, 2, 3- and 4-weeks treatment.

### Cytokine Samples

Plasma samples for CXCL13, IL-1β, IL-6, IL-10, and TNF-α analysis will be taken at baseline, after 2 weeks and 4 weeks treatment.

### Biobanking

The following samples are kept at frozen storage in a biobank or similar, as applicable per country, during the study:
Cytokines (CXCL13, IL-1β, IL-6, IL-10, and TNF-α), plasma
PK, plasma

Analysis of all cytokines and PK will be performed at a central laboratory. The samples will be sent and analyzed on a regular basis.

### Safety

Adverse events (AE) will be monitored from the time that the subject gives informed consent and throughout the study, and will be elicited by direct, non-leading questioning or by spontaneous reports. An AE is any untoward medical occurrence in a subject or clinical investigation subject administered a pharmaceutical product and which does not necessarily have a causal relationship with this treatment. An AE can therefore be any unfavorable and unintended sign (including an abnormal laboratory finding), symptom, or disease temporally associated with the use of a medicinal or investigational product, whether or not considered related to the medicinal or investigational product. Pre-existing conditions which worsen during a study are to be reported as AEs.

### Severity Classification

The severity of an event is evaluated in order to subcategorize events. Severity is not seriousness. A very severe event can be non-serious, and a serious event can be of mild severity. Each event will be graded for severity using Common Terminology Criteria for Adverse Events (CTCAE), v4.03 grading scale: June-4, 2010.

The following grading will apply:
Mild (Grade 1)—Mild; asymptomatic or mild symptoms; clinical or diagnostic observa-ions only; intervention not indicated.
Moderate (Grade 2)—Moderate; minimal, local, or non-invasive intervention indicated; limiting age-appropriate instrumental Activity of Daily Living (ADL).
Severe (Grade 3)—Severe or medically significant but not immediately life-threatening; hospitalization or prolongation of hospitalization indicated; disabling; limiting self-care ADL.
Life-threatening (Grade 4)—Life-threatening consequences; urgent intervention indicated.
Death (Grade 5) related to AE.

A determination will be made of the relationship (if any) between an adverse event and the study drug. A causal relationship is present if a determination is made that there is a reasonable possibility that the adverse event may have been caused by the study drug.

### Example 2

Results from the clinical trial described in Example 1 are presented here. The available data show a significant effect of AP1189 in reducing CDAI for both tested dosages, i.e. 50 mg and 100 mg. Results are indicated at 2 weeks (mid-treatment period), at 4 weeks (end of treatment period), and at 5 weeks (one week follow-up after last dosage).

| | | | | | | | Differ- |
|---|---|---|---|---|---|---|---|
| | Screen- | Base- | Week | Week | Week | Differ- | ence |
| CDAI | ing | line | 2 | 4 | 5 | ence | in % |
| 50 mg | 35.6 | 36.1 | 29.9 | 24.2 | 22.9 | −11.9 | 33% |
| AP1189 | | | | | | | |
| (n = 31) | | | | | | | |
| Group | | | | | | | |
| mean | | | | | | | |
| Standard | 12 | 11.1 | 10.4 | 10.4 | 11.3 | — | — |
| deviation | | | | | | | |
| 100 mg | 38.9 | 39 | 28.9 | 23.9 | 21.3 | −15.1 | 39% |
| AP1189 | | | | | | | |
| (n = 31) | | | | | | | |
| Group | | | | | | | |
| mean | | | | | | | |
| Standard | 9.3 | 9 | 10.9 | 13 | 11.9 | — | — |
| deviation | | | | | | | |
| Placebo | 35.3 | 36.3 | 28.3 | 27.4 | 23.2 | −8.9 | 25% |
| (n = 32) | | | | | | | |
| Group | | | | | | | |
| mean | | | | | | | |
| Standard | 8.4 | 8.6 | 12.3 | 13.6 | 13.8 | — | — |
| deviation | | | | | | | |

The effect of treatment on alanine aminotransferase/alanine transaminase (ALAT) is shown in the below table. With placebo and at a dosage of 50 mg AP1819 an increase in ALAT is observed, at both 2 weeks, 4 weeks (end of treatment, EoT) and 5 weeks (one week after final dosage; follow-on visit FoV). ALAT is commonly elevated following MTX treatment.

At a dosage of 100 mg AP1189 no ALAT elevation is observed (0.0%).

| ALAT | 2 weeks | n | CSA |
|---|---|---|---|
| Placebo | 3.1% | 32 | 1 |
| 50 mg AP1189 | 6.3% | 32 | 2 |
| 100 mg AP1189 | 0.0% | 34 | 0 |

CSA = Clinical significant abnormal value

| ALAT | 4 weeks | n | CSA |
|---|---|---|---|
| Placebo | 6.3% | 32 | 2 |
| 50 mg AP1189 | 6.1% | 33 | 2 |
| 100 mg AP1189 | 0.0% | 33 | 0 |

| ALAT | 5 weeks | n | CSA |
|---|---|---|---|
| Placebo | 3.0% | 33 | 1 |
| 50 mg AP1189 | 6.3% | 32 | 2 |
| 100 mg AP1189 | 0.0% | 35 | 0 |

The baseline characteristics of the study participants are comparable between the groups:

| | | AP1189 | AP1189 |
|---|---|---|---|
| | Placebo | 50 mg | 100 mg |
| | (n = 34) | (n = 35) | (n = 35) |
| Age | 56.4 ± 13.1 | 56.4 ± 13.1 | 55.3 ± 13.9 |
| | 61 (range: | 57 (range: | 56.5 (range: |
| | 26-78) | 28-79) | 27-77) |
| Age 18-64: | 73.5% | 71.4% | 72.2% |
| Age 65-84: | 26.5% | 28.5% | 27.8% |
| Weight | 75.2 ± 19.7 | 75.9 ± 17.7 | 79.3 ± 16.7 |
| | 71.6 (range: | 75.3 (range: | 76.6 (range: |
| | 48-145) | 42-111) | 48-118) |
| Height | 167.5 ± 7.8 | 167.5 ± 6.6 | [symbol] ± 8.4 |
| | 167 (range: | 167 (range: | 165.5 (range: |
| | 155-185) | 157-183) | 151-185) |
| Ethnicity | 34 White | 34 White, 1 Asian | 35 White, 1 Other |
| Gender | 79.4% Females | 77.1% Females | 77.8% Females |

| | | AP1189 | AP1189 |
|---|---|---|---|
| | Placebo | 50 mg | 100 mg |
| | (n = 32) | (n = 31)* | (n = 35)* |
| Clinical Disease | 36.3 ± 8.6 | 36.1 ± 11.0 | 39 ± 9 |
| Activity Index (CDAI): | | | |
| QVAS-Patient: | 6.2 ± 1.2 | 6.3 ± 1.3 | 6.4 ± 1.3 |
| QVAS-Investigator: | 6.6 ± 1.8 | 5.9 ± 2.3 | 5.8 ± 2.1 |
| Swollen Joints (century) | 10.0 ± 3.9 | 10.3 ± 4.4 | 11.8 ± 5.0 |
| Tender Joints (century) | 13.9 ± 5.6 | 13.7 ± 6.4 | 15.0 ± 5.8 |
| DAS28 Score | 5.5 ± 1.0 | 5.4 ± 0.9 | 5.7 ± 0.9 |
| CRP (mg/L) | 19.1 ± 26.5 | 14.9 ± 18.4 | 26.1 ± 40.7 |

### REFERENCES

1. Perretti et al. Trends Pharmacol Sci. 36:737-55, 2015
2. Brzoska et al. Endocr Rev. 29:581-602, 2008
3. Montero-Melendez et al. Am J Pathol. 179:259-69, 2011
4. Catania et al., 2004
5. Garcia-Borron et al. Pigment Cell Res 18:393-410, 2005
6. Slominski et al., 2000
7. Hunt et al., 1994
8. Abdel-Malek et al., 1995
9. Abdel-Malek, 2001
10. Manna and Aggarwal, 1998
11. Buckley and Ramachandran, 1981
12. Mountjoy et al., 1992
13. Xia and Wikberg, 1996
14. Boston, 1999
15. Chhajlani, 1996
16. Gantz et al., 1993
17. Roselli-Rehfuss et al., 1993
18. Getting, 2002
19. Li et al., 1996
20. Chen et al., 2000
21. Guarini et al., 2002
22. Getting and Perretti, 2000
23. Alvaro et al., 1996
24. Mountjoy et al., 1994
25. Marsh et al., 1999
26. Van der Ploeg et al., 2002
27. Taylor and Namba, 2001
28. Chen et al., 1997
29. Thody and Shuster, 1970
30. Lindskog et al., 2010.
31. Silman A J, Pearson J E, Epidemiology and genetics of rheumatoid arthritis. Arthritis Res. 2002; 4 Suppl 3: S265-72. Epub 2002 May 9
32. Gibofsky A. Overview of epidemiology, pathophysiology, and diagnosis of rheumatoid arthritis. Am J Manag Care 2012; 18: S295-302
33. Blumberg S, Fox D. Rheumatoid Arthritis: Guidelines for Emerging Therapies. Am J Manag Care. 2001; 7 (6): 617-26.
34. Hirano T. The biology of interleukin-6. Chem immunol. 1992; 51:153-180
35. Keller T K, Wanagat J, Erschler W B. Molecular and cellular biology of interleukin-6 and its receptor. Frontiers Biosci. 1996; 1:340-357
36. Metzger S, Hassin T, Barash V, Pappo O, Chajek-Shaul T. Reduced body fat and increased hepatic lipid synthesis in mice bearing interleukin-6-secreting tumor. Am J Physiol Endocrinol Metab. 2001; 281: E597-E965
37. Tamura T, Udagawa N, Takahashi N, Miyaura C, Tanaka S, Yamada Y, et al. Soluble interleukin-6 receptor triggers osteoclast formation by interleukin-6. Proc Natl Acad Sci USA. 1993; 90:11924-11928
38 Taub R. Hepatoprotection via the IL-6/Stat3 pathway. J Clin Invest 2003; 112:978-980
39. Hirano T, Matsuda T, Turner M, Miyasaka N, Buchan G, Tang B, et al. Excessive production of IL-6/B cell stimulatory factor-2 in rheumatoid arthritis. Eur J Immunol. 1988; 18:1797-1801
40 Houssiau F A, Devogelaer J P, Van Damme J, de Deuxchaisnes C N, Van Snick J. IL-6 in synovial fluid and serum of patients with rheumatoid arthritis and other inflammatory arthritides. Arthritis Rheum. 1988; 31:784-788
41. Madhok R, et al. The effect of second line drugs on serum interleukin 6 levels in rheumatoid arthritis. Arthritis Rheum. 1990; 33: S154. Abstract
42. Salaffi F1, Cimmino M A, Leardini G, Gasparini S, Grassi W. Disease activity assessment of rheumatoid arthritis in daily practice: validity, internal consistency, reliability and congruency of the Disease Activity Score including 28 joints (DAS28) compared with the Clinical Disease Activity Index (CDAI). 2009 July-August; 27 (4): 552-9
43. https://doi.org/10.1161/JAHA. 116.003264. Journal of the American Heart
44. Association. 2016; 5: e003264. Originally published Jun. 17, 2016 Rooney M, Whelan A, Feighery C, Bresnihan B Changes in lymphocyte infiltration of the synovial membrane and the clinical course of rheumatoid arthritis. Arthritis Rheum. 1989; 32:361-369
45. Walters M T, Smith J L, Moore K, Evans P R, Cawley M I D An investigation of the action of disease modifying anti-rheumatic drugs on the rheumatoid synovial membrane: reduction in T lymphocyte subpopulations and HLA-DP and DQ antigen expression after gold or penicillamine therapy. Ann Rheum Dis 1987; 30:1-10
46. Firestein G S, Paine M M, Littman B H Gene expression (collagenase, tissue inhibitor of metalloproteinases, complement and HLA-DR) in rheumatoid arthritis synovium. Quantitative analysis and effect of intra-articular corticosteroids. Arthritis Rheum 1991; 34:1094-1105
47. Firestein G S, Paine M M, Boyle D L Mechanism of methotrexate action in rheumatoid arthritis. Selective decrease in synovial collagenase gene expression. Arthritis Rheum 1994; 37:193-200
48. Yanni G, Farahat MNMR, Poston R N, Panayi G S Intramuscular gold decreases cytokine expression and macrophage numbers in the rheumatoid synovial membrane. Ann Rheum Dis 1994; 53:315-322
49. Youssef P P, Cormack J, Evill C A, Peter D T, Roberts-Thompson P J, Ahern M J, et al. Neutrophil trafficking into inflamed joints in patients with rheumatoid arthritis and the effect of methylprednisolone. Arthritis Rheum 1996; 39:236-242
50. Youssef P P, Haynes D R, Triantafillou S, Parker A, Gamble J R, Roberts-Thomson P J, et al. Effects of pulse methylprednisolone on inflammatory mediators in peripheral blood, synovial fluid, and synovial membrane in rheumatoid arthritis. Arthritis Rheum 1997; 40:1400-1408
51 Dolhain RJEM, Tak P P, Dijkmans B A C, de Kuiper P, Breedveld F C, Miltenburg A M M Methotrexate treatment reduced inflammatory cell numbers, expression of monokines and of adhesion molecules in synovial tissue of patients with rheumatoid arthritis. Br J Rheumatol 1998; 37:502-508
52. Bresnihan B, Tak P P Synovial tissue analysis in rheumatoid arthritis. Ballieres Clin Rheumatol 1999; 13:645-659
53. Non-Clinical Safety Studies for the Conduct of Human Clinical Trials for Pharmaceuticals (M3, rev 1). International Conference on Harmonization. Current Step 4 version, dated 9 Nov. 2000.
54. Sergeant et al. Arthritis Research & Therapy (2018) 20:147).

1. A kit of parts comprising
a. more than 41.5 mg, such as at least about 50 mg, such as at least about 62 mg, such as at least 75 mg of a compound of formula (I): 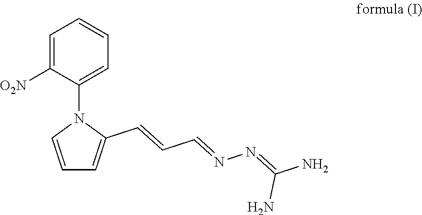 [SMILES: NC(N)=N/N=C/C=C/c1cccn1-c1ccccc1[N+](=O)[O-]]
including tautomeric and stereoisomeric forms thereof; or a pharmaceutically acceptable derivative thereof,
for administration daily, such as once daily, and
b. 5 to 30 mg methotrexate (MTX), for administration once weekly.

2. The kit of parts according to claim 1, further comprising
c. Folic acid, such as about 1 to about 10 mg folic acid for administration per week, such as about 5 mg folic acid for administration per week, such as at least 5 mg folic acid for administration per week.

3. The kit of parts according to claim 2, wherein said folic acid is for administration once weekly, or wherein said folic acid is for administration in daily dosages.

4. The kit of parts according to any of the preceding claims, wherein said kit of parts is for use in the treatment of rheumatoid arthritis.

5. A compound of formula (I) 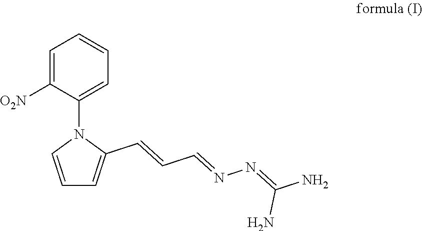 [SMILES: NC(N)=N/N=C/C=C/c1cccn1-c1ccccc1[N+](=O)[O-]]
including tautomeric and stereoisomeric forms thereof; or a pharmaceutically acceptable derivative thereof,
for use in the treatment of rheumatoid arthritis,
wherein said compound is to be administered with methotrexate (MTX),
wherein said compound is administered at a dosage of more than 41.5 mg daily, such as at least about 50 mg daily, such as at least about 62 mg daily, such as at least about 75 mg daily, such as once daily,
and said MTX is administered at a dosage of 5 to 30 mg once weekly.

6. A combination of methotrexate (MTX) and a compound of formula (I) 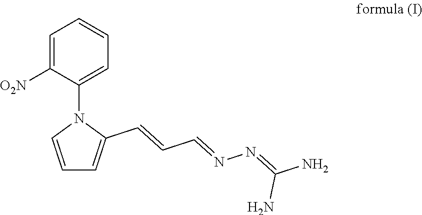 [SMILES: NC(N)=N/N=C/C=C/c1cccn1-c1ccccc1[N+](=O)[O-]]
including tautomeric and stereoisomeric forms thereof; or a pharmaceutically acceptable derivative thereof,
for use in the treatment of rheumatoid arthritis,
wherein said compound is administered at a dosage of more than 41.5 mg daily, such as at least about 50 mg daily, such as at least about 62 mg daily, such as at least about 75 mg daily, such as once daily,
and said MTX is administered at a dosage of 5 to 30 mg once weekly.

7. The kit of parts, the compound for use or the combination for use according to any of the preceding claims, wherein said compound is selected from the group consisting of {3-[1-(2-nitrophenyl)-1H-pyrrol-2-yl]-allylidene}-aminoguanidine and (E)-N-trans-{3-[1-(2-nitrophenyl)-1H-pyrrol-2-yl]-allylidene}-aminoguanidine, or a pharmaceutically acceptable derivative thereof.

8. The kit of parts, the compound for use or the combination for use according to any of the preceding claims, wherein said pharmaceutically acceptable derivative is a pharmaceutically acceptable salt of an inorganic acid or an organic acid.

9. The kit of parts, the compound for use or the combination for use according to any of the preceding claims, wherein said compound is (E)-N-trans-{3-[1-(2-nitrophenyl)-1H-pyrrol-2-yl]-allylidene}-aminoguanidine, or a pharmaceutically acceptable salt thereof.

10. The kit of parts, the compound for use or the combination for use according to any of the preceding claims, wherein said organic acid is selected from the group consisting of: formic acid, acetic acid, trichloroacetic acid, trifluoroacetic acid, propionic acid, benzoic acid, cinnamic acid, citric acid, fumaric acid, glycolic acid, lactic acid such as L-lactic acid or DL-lactic acid, maleic acid, malic acid, malonic acid, mandelic acid such as DL-mandelic acid, oxalic acid, picric acid, pyruvic acid, salicylic acid, succinic acid, methanesulfonic acid, ethanesulfonic acid, tartaric acid, ascorbic acid, pamoic acid, bismethylene salicylic acid, ethanedisulfonic acid, gluconic acid, citraconic acid, aspartic acid, stearic acid, palmitic acid, EDTA, glycolic acid, p-aminobenzoic acid, glutamic acid, benzenesulfonic acid, p-toluenesulfonic acid hippuric acid, oxoglutaric acid such as 2-oxoglutaric acid or 3-oxoglutaric acid, glutaric acid, and adipic acid.

11. The kit of parts, the compound for use or the combination for use according to any of the preceding claims, wherein said organic acid is selected from the group consisting of acetic acid, succinic acid, tartaric acid or propionic acid.

12. The kit of parts, the compound for use or the combination for use according to any of the preceding claims, wherein said organic acid is selected from the group consisting of acetic acid and succinic acid.

13. The kit of parts, the compound for use or the combination for use according to any of the preceding claims, wherein said organic acid is acetic acid.

14. The kit of parts, the compound for use or the combination for use according to any of the preceding claims, wherein said organic acid is succinic acid.

15. The kit of parts, the compound for use or the combination for use according to any of the preceding claims, wherein said inorganic acid is selected from the group consisting of: hydrochloric acid, hydrobromic acid, hydroiodic acid, phosphoric acid, sulphuric acid and nitric acid.

16. The kit of parts, the compound for use or the combination for use according to any of the preceding claims, wherein said compound is selected from the group consisting of {3-[1-(2-nitrophenyl)-1H-pyrrol-2-yl]-allylidene}-aminoguanidinium acetate and (E)-N-trans-{3-[1-(2-nitrophenyl)-1H-pyrrol-2-yl]-allylidene}-aminoguanidinium acetate.

17. The kit of parts, the compound for use or the combination for use according to any of the preceding claims, wherein said compound is selected from the group consisting of {3-[1-(2-nitrophenyl)-1H-pyrrol-2-yl]-allylidene}-aminoguanidinium succinate and (E)-N-trans-{3-[1-(2-nitrophenyl)-1H-pyrrol-2-yl]-allylidene}-aminoguanidinium succinate.

18. The kit of parts, the compound for use or the combination for use according to any of the preceding claims, wherein said compound is selected from the group consisting of (E)-N-trans-{3-[1-(2-nitrophenyl)-1H-pyrrol-2-yl]-allylidene}-aminoguanidinium acetate and (E)-N-trans-{3-[1-(2-nitrophenyl)-1H-pyrrol-2-yl]-allylidene}-aminoguanidinium succinate.

19. The kit of parts, the compound for use or the combination for use according to any of the preceding claims, wherein said compound is (E)-N-trans-{3-[1-(2-nitrophenyl)-1H-pyrrol-2-yl]-allylidene}-aminoguanidinium acetate.

20. The kit of parts, the compound for use or the combination for use according to any of the preceding claims, wherein said compound is (E)-N-trans-{3-[1-(2-nitrophenyl)-1H-pyrrol-2-yl]-allylidene}-aminoguanidinium succinate.

21. The kit of parts, the compound for use or the combination for use according to any of the preceding claims, wherein said compound comprises amorphous forms and polymorphic (crystalline) forms.

22. The kit of parts, the compound for use or the combination for use according to any of the preceding claims, wherein said compound is administered at a dosage of more than 41.5 mg daily (calculated as the free base).

23. The kit of parts, the compound for use or the combination for use according to any of the preceding claims, wherein said compound is administered at a dosage of at least 42 mg, such as at least 43 mg, such as at least 44 mg, such as at least about 45 mg, such as at least about 46 mg, such as at least about 47 mg, such as at least about 48 mg, such as at least about 49 mg (calculated as the free base).

24. The kit of parts, the compound for use or the combination for use according to any of the preceding claims, wherein said compound is administered at a dosage of at least about 50 mg daily (calculated as the free base).

25. The kit of parts, the compound for use or the combination for use according to any of the preceding claims, wherein said compound is administered at a dosage of at least about 62 mg daily (calculated as the free base).

26. The kit of parts, the compound for use or the combination for use according to any of the preceding claims, wherein said compound is administered at a dosage of at least about 75 mg daily (calculated as the free base).

27. The kit of parts, the compound for use or the combination for use according to any of the preceding claims, wherein said compound is administered at a daily dosage of more than 41.5 mg per day to about 250 mg per day (calculated as the free base), such as about 42 to about 45 mg per day, about 45 to about 50 mg per day, about 50 to about 55 mg, about 55 to about 60 mg, about 60 to about 62 mg, about 62 to about 65 mg, about 65 to about 70 mg, such as about 70 to about 75 mg per day, about 75 to about 80 mg, about 80 to about 83 mg, about 83 to about 85 mg, about 85 to about 90 mg, about 90 to about 95 mg, about 95 to about 100 mg, about 100 to about 125 mg, about 125 to about 150 mg, about 150 to about 166 mg, about 166 to about 175 mg, about 175 to about 200 mg, such as about 200 to about 250 mg per day (calculated as the free base).

28. The kit of parts, the compound for use or the combination for use according to any of the preceding claims, wherein said compound is administered at a dosage of at least 83 mg daily (calculated as the free base).

29. The kit of parts, the compound for use or the combination for use according to any of the preceding claims, wherein said compound is the acetate salt and is administered at a dosage of at least about 75 mg daily (calculated as the acetate salt).

30. The kit of parts, the compound for use or the combination for use according to any of the preceding claims, wherein said MTX is selected from the group consisting of methotrexate (systemic), methotrexate (oral), methotrexate tablet, methotrexate oral solution, methotrexate (injection), methotrexate sodium, Methotrexate LPF Sodium, Trexall (Xatmep), Rheumatrex, Rasuvo, Otrexup, Alltrex, Beltrax, Biotrexate, Caditrex, Carditrex, Cytotrex, Dermotrex, Folitrax, HI-Trex, Imutrex, Merex, Methocip, Methorex, Methotrexate, Metorex, Metrex, Mexate, MTX-Korea, Neotrexate, Nidtrex, Oncotrex, Onotrex, Plastomet, Remtrex, Rextop, Roxate, Tevatrex, Throtex, Trex, Thixilem, Vibzi and Zexate.

31. The kit of parts, the compound for use or the combination for use according to any of the preceding claims, wherein said MTX is administered at a dosage of about 5 mg to about 30 mg once weekly.

32. The kit of parts, the compound for use or the combination for use according to any of the preceding claims, wherein said MTX is administered at a dosage of about 5 mg once weekly, such as about 7.5 mg once weekly, such as about 10 mg once weekly, such as about 15 mg once weekly, such as about 20 mg once weekly, such as about 25 mg once weekly, such as about 30 mg once weekly.

33. The kit of parts, the compound for use or the combination for use according to any of the preceding claims, wherein said MTX is administered at a dosage of about 10 mg to about 25 mg once weekly.

34. The compound for use or the combination for use according to any of the preceding claims, wherein said compound is to be administered with folic acid, or an equivalent thereof.

35. The compound for use or the combination for use according to any of the preceding claims, further comprising one or more steps of administering folic acid, or an equivalent thereof.

36. The compound for use or the combination for use according to any of the preceding claims, further comprising administering about 1 to 10 mg folic acid per week, such as about 5 mg folic acid per week, such as at least 5 mg folic acid per week.

37. The kit of parts, the compound for use or the combination for use according to any of the preceding claims, wherein said folic acid is administered once weekly, or wherein said folic acid is administered in daily dosages.

38. The kit of parts, the compound for use or the combination for use according to any of the preceding claims, wherein treatment with MTX and said compound is initiated essentially at the same time.

39. The kit of parts, the compound for use or the combination for use according to any of the preceding claims, wherein treatment with MTX is initiated prior to treatment with said compound.

40. The kit of parts, the compound for use or the combination for use according to any of the preceding claims, wherein the subject with rheumatoid arthritis is naîve to MTX treatment prior to initiating treatment with the compound.

41. The kit of parts, the compound for use or the combination for use according to any of the preceding claims, wherein elevation of one or more aminotransferases is reduced or abolished, such as wherein elevation of alanine aminotransferase/alanine transaminase (ALAT) is reduced or abolished.

42. The kit of parts, the compound for use or the combination for use according to any of the preceding claims, wherein MTX-induced elevation of one or more aminotransferases is reduced or abolished, such as wherein MTX-induced elevation of alanine aminotransferase/alanine transaminase (ALAT) is reduced or abolished.

43. The kit of parts, the compound for use or the combination for use according to any of the preceding claims, wherein MTX-induced elevation of aminotransferases, such as alanine aminotransferase/alanine transaminase (ALAT), does not occur and/or is not observed.

44. The kit of parts, the compound for use or the combination for use according to any of the preceding claims, wherein elevation of one or more aminotransferases, such as MTX-induced elevation of one or more aminotransferases, such as ALAT, is less than 3%.

45. The kit of parts, the compound for use or the combination for use according to any of the preceding claims, wherein elevation of one or more aminotransferases, such as MTX-induced elevation of one or more aminotransferases, such as ALAT, is less than 6%.

46. The kit of parts, the compound for use or the combination for use according to any of the preceding claims, wherein elevation of one or more aminotransferases, such as MTX-induced elevation of one or more aminotransferases, such as ALAT, is about 0%, such as about 1% or less, such as about 2% or less, such as less than 3%, such as about 3% or less, such as about 4% or less, such as about 5% or less, such as less than about 6%, such as about 6% or less.

47. The kit of parts, the compound for use or the combination for use according to any of the preceding claims, wherein no, or essentially no, elevation of aminotransferases, such as alanine aminotransferase/alanine transaminase (ALAT), occurs and/or is observed.

48. The kit of parts, the compound for use or the combination for use according to any of the preceding claims, wherein said rheumatoid arthritis is severe active rheumatoid arthritis.

49. The kit of parts, the compound for use or the combination for use according to any of the preceding claims, wherein said rheumatoid arthritis is rheumatoid arthritis with a CDAI>22.

50. The kit of parts, the compound for use or the combination for use according to any of the preceding claims, wherein said rheumatoid arthritis is early rheumatoid arthritis with active joint disease.

51. The kit of parts, the compound for use or the combination for use according to any of the preceding claims, wherein said rheumatoid arthritis is newly diagnosed subjects with severe active rheumatoid arthritis (CDAI>22) who are to start up-titration with MTX.

52. The kit of parts, the compound for use or the combination for use according to any of the preceding claims, wherein said rheumatoid arthritis is newly diagnosed subjects with rheumatoid arthritis (RA) with an inadequate response (IR) to MTX therapy.

53. The kit of parts, the compound for use or the combination for use according to any of the preceding claims, wherein said rheumatoid arthritis is rheumatoid arthritis in a subject with an inadequate response to MTX therapy.

54. The kit of parts, the compound for use or the combination for use according to any of the preceding claims, wherein said rheumatoid arthritis is DMARD-inadequate response rheumatoid arthritis.

55. The kit of parts, the compound for use or the combination for use according to any of the preceding claims, wherein the subject's CDAI score during and/or after treatment result in a 5-point decrease, such as a 10-point decrease, such as a 15-point decrease in the subjects CDAI.

56. The kit of parts, the compound for use or the combination for use according to any of the preceding claims, wherein the CDAI score is reduced by at least 2 points, such as at least 3 points, such as at least 4 points, such as at least 5 points, such as at least 6 points, such as at least 7 points, such as at least 8 points, such as at least 9 points, such as at least 10 points, such as at least 11 points, such as at least 12 points, such as at least 13 points, such as at least 14 points, such as at least 15 points, such as at least 16 points, such as at least 17 points, such as at least 18 points, such as at least 19 points, such as at least 20 points.

57. The kit of parts, the compound for use or the combination for use according to any of the preceding claims, wherein the reduced CDAI score is indicated at week 2 after treatment initiation; or is indicated at week 4 after treatment initiation; or is indicated at end of treatment period; or is indicated at week 5 after treatment initiation; or is indicated at one week follow-up after last dosage.

58. The kit of parts, the compound for use or the combination for use according to any of the preceding claims, wherein said rheumatoid arthritis is rheumatoid arthritis with a DAS28 score of above 5.1.

59. The kit of parts, the compound for use or the combination for use according to any of the preceding claims, wherein said compound is formulated in a pharmaceutical composition or pharmaceutical formulation, such as an oral formulation.

60. The kit of parts, the compound for use or the combination for use according to any of the preceding claims, wherein said compound is formulated as a powder, such as a powder for suspension.

61. The kit of parts, the compound for use or the combination for use according to any of the preceding claims, wherein said compound is formulated in an oral suspension, such as an alkaline oral suspension.

62. The kit of parts, the compound for use or the combination for use according to any of the preceding claims, wherein said compound is formulated in a solid oral formulation such as a solid oral dosage form.

63. The kit of parts, the compound for use or the combination for use according to any of the preceding claims, wherein said compound is formulated in a tablet, such as an immediate release tablet.

64. The kit of parts, the compound for use or the combination for use according to any of the preceding claims, wherein said oral formulation delivers or releases said compound to the gastric compartment (or stomach), such as primarily or predominantly delivers or releases said compound to the gastric compartment (or stomach).

65. The kit of parts, the compound for use or the combination for use according to any of the preceding claims, wherein said oral formulation delivers or releases not less than about 65% to about 80% of said compound in the gastric compartment; such as not less than about 65% of said compound, such as not less than about 70%, such as not less than about 75%, such as not less than about 80%, such as not less than about 85%, such as not less than about 90%, such as not less than about 95% of said compound in the gastric compartment.

66. The kit of parts, the compound for use or the combination for use according to any of the preceding claims, wherein not less than about 65% of said compound, such as not less than about 70%, such as not less than about 75%, such as not less than about 80%, such as not less than about 85%, such as not less than about 90%, such as not less than about 95% of said compound is dissolved into solution in the gastric compartment.

67. The kit of parts, the compound for use or the combination for use according to any of the preceding claims, wherein not less than about 65% to about 80% of said compound is dissolved into solution in about 5 minutes at a pH of about 1 to 3, such as a pH of about 0.5 to 3.5; such as a pH of about 0.5 to 1, such as a pH of about 1 to 1.5, such as a pH of about 1.5 to 2, such as a pH of about 2 to 2.5, such as a pH of about 2.5 to 3, such as a pH of about 3 to 3.5.
